(19) United States
(12) Patent Application Publication
(10) Pub. No.: US 2025/0018247 A1
(43) Pub. Date: Jan. 16, 2025

(54) SYSTEMS AND METHODS FOR ATHLETIC ACTIVITY DATA VERIFICATION AND VIRTUAL POINT ALLOCATION

(71) Applicant: NIKE, Inc., Beaverton, OR (US)

(72) Inventors: Yana Baradyntsava, Amsterdam (NL); Thomas Castelijns, Breda (NL); Vivianne Driessen, Amsterdam (NL); Thomas Hantke, Amsterdam (NL); Dzmitry Kankalovich, Haarlem (NL); Nima Maleki, Uithoorn (NL); Mark McDermont, London (GB); Joep Serrarens, Amsterdam (NL); Gijs Wobben, Amsterdam (NL)

(21) Appl. No.: 18/741,983

(22) Filed: Jun. 13, 2024

Related U.S. Application Data
(60) Provisional application No. 63/521,124, filed on Jun. 15, 2023.

(51) Int. Cl. A63B 24/00

(52) U.S. Cl. CPC A63B 24/0062; A63B 2024/0068

(57) ABSTRACT

Systems and methods described herein receiving, from an activity monitoring device associated with a user, an indication that the user is to perform a first athletic activity, determining a user profile associated with the user for an athletic activity virtual point system, receiving activity information as the user performs a first athletic activity, verifying the first athletic activity including determining that the activity information falls within one or more thresholds associated with the user that are determined based on the user profile, determining a first amount of virtual activity points to allocate to the user profile based on the first athletic activity, allocating the first amount of virtual activity points to the user profile, and providing a virtual activity point display showing the first amount virtual activity points accumulated from the first athletic activity and one or more aspects of the first athletic activity.

### RELATED APPLICATION DATA

This application is a U.S. Non-Provisional Application and claims priority benefits based on U.S. Provisional Patent Application. No. 63/521,124, filed Jun. 15, 2023, which is incorporated by reference herein in its entirety.

### TECHNICAL FIELD

Aspects of the present disclosure generally relate to systems, apparatuses, and methods for monitoring, analyzing, and tracking athletic activity data and, more particularly, to systems, apparatuses, and methods for processing and analyzing athletic activity data and providing a virtual reward system based on analyzed athletic activity data.

### BACKGROUND

While most people appreciate the importance of physical fitness, many have difficulty finding the motivation required to maintain a regular exercise program. Some people find it particularly difficult to maintain an exercise regimen that involves continuously repetitive motions, such as running, walking and bicycling. Experienced athletes and trainers have found that feedback provides many people with motivation to maintain a regular exercise program. When a person can directly experience the results provided by an exercise program, that person typically will be encouraged to continue exercising.

Additionally, individuals may view exercise as work or a chore and thus, separate it from enjoyable aspects of their daily lives. This clear separation between athletic activity and other activities reduces the amount of motivation that an individual might have toward exercising. Athletic activity services and systems directed toward encouraging individuals to engage in athletic activities might also be too focused on one or more particular activities while an individual's interests are ignored. This may further decrease a user's interest in participating in athletic activities or using the athletic activity services and systems. Additionally, individuals may sometimes look for ways to game the system, e.g., by not actually performing a particular workout or by performing an activity with low effort, and thereby reducing the positive effects of certain activity reward systems.

Therefore, improved systems and methods to address these and other shortcomings in the art are desired.

### SUMMARY

In light of the foregoing background, the following presents a simplified summary of the present disclosure in order to provide a basic understanding of some aspects of the disclosure. This summary is not an extensive overview of the disclosure. It is neither intended to identify key or critical elements of the disclosure nor to delineate the scope of the disclosure. The following summary merely presents some concepts of the disclosure in a simplified form as a prelude to the more detailed description provided below.

Aspects of this disclosure relate to processing and analyzing athletic activity data and providing a virtual reward system based on analyzed athletic activity data. In accordance with aspects of the present disclosure, apparatuses, systems, and methods described herein provide for efficient processing and analyzing of athletic activity data and virtual reward systems based on analyzed athletic activity data.

According to certain aspects of the present disclosure, a method is provided that includes receiving, by a processor, a user request to set up a user profile for an athletic activity virtual point system, wherein the user request includes user profile information, and receiving, from an activity monitoring device associated with the user, activity information as the user performs a first athletic activity. The method may then include verifying the first athletic activity, wherein verifying the first athletic activity includes determining that the activity information falls within one or more thresholds associated with the user that are determined based on the user profile information. Thereafter, upon verifying the first athletic activity, the method may include determining a first amount of virtual activity points to allocate to the user profile based on the first athletic activity. Subsequently, the method may include allocating the first amount of virtual activity points to the user profile, and providing a virtual activity point display showing the first virtual activity points accumulated from the first athletic activity and one or more aspects of the first athletic activity.

In one or more instances, receiving user profile information may include receiving benchmark activity data, from the activity monitoring device, as the user performs a benchmark activity. The one or more thresholds for the user may be determined based on the benchmark activity data. In some instances, receiving user profile information may include receiving a plurality of user input parameters. The plurality of user input parameters may include one or more of: age, sex, height, weight, medical issue, pace, heart rate, athletic equipment identification, target athletic goal, and personal record. The one or more thresholds for the user may be determined based on at least one of the plurality of user input parameters. In some instances, the method may further include training a machine learning model to determine the one or more thresholds for the user based on the plurality of user input parameters and accumulated data of a plurality of users across various user demographics. The one or more thresholds for the user may be updated based on activity information as the user performs one or more athletic activities.

In one or more instances, verifying the first athletic activity may include training a machine learning model to identify anomalous activity associated with the user profile based on at least the user profile information. In some instances, the method may further include authenticating the user associating with the user profile before the user begins the first athletic activity. Authenticating the user may include presenting a photo prompt to a display of a user device, receiving a photo responsive to user interaction with the photo prompt, and verifying, using an image recognition algorithm, that the photo matches image characteristics of the user associated with the user profile within a threshold amount. In some instances, verifying the first athletic activity may include, upon determining that the activity information falls outside of the one or more thresholds associated with the user, determining a likelihood of anomalous data based on at least one of: a type of anomaly in the activity information or an extent to which the activity information falls outside of the one or more thresholds.

In one or more instances, determining a first amount of virtual activity points may include determining one or more parameters associated with the first athletic activity selected from: a level of effort by the user while performing the first athletic activity, a type of activity of the first athletic activity, and a time for the user to complete the first athletic activity, and selecting, based on the one or more parameters associated with the first athletic activity, a conversion rate from a plurality of rates for converting activity data to virtual activity points. In some instances, determining a first amount of virtual activity points may include determining a level of effort by the user while performing the first athletic activity, and comparing the level of effort by the user while performing the first athletic activity to a baseline effort of the user. The baseline effort of the user may be determined by training a machine learning model based on data from the user as the user performs one or more previous athletic activities and/or based on user profile information.

In one or more instances, the method may further include providing a workout display to a user device during the first athletic activity. The workout display may include an estimate of activity points accumulated during the first athletic activity. The virtual activity point display may include a detailed activity point breakdown including with one or more parameters associated with the first athletic activity, one or more sections of the first athletic activity, and allotted points for each of the one or more sections.

In one or more instances, the method may further include upon the user interacting with a prompt to begin a second athletic activity, determining if the user has exceeded a cap on virtual activity points, and upon determining that the user has exceeded the cap, issuing a notification to the user indicating that virtual activity points will not be allotted for the second workout.

In one or more instances, the method may further include converting virtual activity points of the user profile to a currency, presenting a marketplace display to the user device with an indication of virtual points for items in the marketplace display, and upon receiving a user interaction with an item in the marketplace display, transmitting an item order for the user and removing an associated amount of virtual activity points from the user profile. In some instances, the method may further include upon determining that the activity information falls outside of one or more thresholds associated with the user, determining if the activity information is indicative of an anomaly relative to the user profile, and issuing an alert to the user device indicating that the activity information is indicative of an anomaly and prompting the user for a follow up verification. In some instances, the method may further include determining if the user has missed a scheduled activity, and issuing a prompt to the user device for the user to perform one or more replacement activities, wherein the one or more replacement activities are selected based on being associated with a potential virtual point allotment in proportion to a potential virtual point allotment of the missed scheduled activity.

According to certain aspects of the present disclosure, a non-transitory computer-readable medium storage medium is provided, storing computer readable instructions that, when executed, cause a processor to perform a method. The method may include: receiving, from an activity monitoring device associated with a user, an indication that the user is to perform a first athletic activity, determining a user profile associated with the user for an athletic activity virtual point system, receiving, from the activity monitoring device, activity information as the user performs a first athletic activity, verifying the first athletic activity, wherein verifying the first athletic activity includes determining that the activity information falls within one or more thresholds associated with the user that are determined based on the user profile, upon verifying the first athletic activity, determining a first amount of virtual activity points to allocate to the user profile based on the first athletic activity, allocating the first amount of virtual activity points to the user profile, and providing, to a display of the activity monitoring device, a virtual activity point display showing the first virtual activity points accumulated from the first athletic activity and one or more aspects of the first athletic activity.

In certain examples, verifying the first athletic activity may include training a machine learning model to determine the one or more thresholds for the user based on a plurality of user input parameters. In some aspects, verifying the first athletic activity may include includes, upon determining that the activity information falls outside of the one or more thresholds associated with the user, determining a likelihood of anomalous data based on at least one of: a type of anomaly in the activity information or an extent to which the activity information falls outside of the one or more thresholds.

According to certain aspects of the present disclosure, a system is provided that includes an activity monitoring device configured to capture activity data relating to an activity performed by a user, and an electronic device in communication with the athletic activity monitoring. The electronic device may include a processor and a non-transitory computer-readable storage medium storing computer-executable instructions that, when executed, cause a processor to perform a method. The method may include: receiving, by the processor, user profile information as part of setting up a user profile for an athletic activity virtual point system; receiving, from the activity monitoring device associated with the user, activity information as the user performs a first athletic activity; verifying the first athletic activity, wherein verifying the first athletic activity includes determining that the activity information falls within one or more thresholds associated with the user that are determined based on the user profile information; upon verifying the first athletic activity, determining a first amount of virtual activity points to allocate to the user profile based on the first athletic activity; and providing, to a display of the electronic device, a virtual activity point display showing the first virtual activity points accumulated from the first athletic activity and one or more aspects of the first athletic activity.

The arrangements described can also include other additional elements, steps, computer-executable instructions, or computer-readable data structures. In this regard, other embodiments are disclosed and claimed herein as well. The details of these and other aspects of the present disclosure are set forth in the accompanying drawings and the description below. Other features and advantages of the present disclosure will be apparent from the description, drawings, and claims.

### BRIEF DESCRIPTION OF THE DRAWINGS

The present disclosure is illustrated by way of example and is not limited by the accompanying figures in which like reference numerals indicate similar elements and in which:

FIG. 1 illustrates an operating environment in which one or more aspects of the present disclosure may be implemented;

FIGS. 2A and 2B illustrate an example of an athletic activity monitoring device that may be employed according to one or more aspects of the present disclosure;

FIG. 3 illustrates an example sensor assembly that may be worn by a user in accordance with one or more aspects of the present disclosure;

FIG. 4 illustrates another example sensor assembly that may be worn by a user in accordance with one or more aspects of the present disclosure;

FIG. 5 schematically illustrates an example article of footwear in accordance with one or more aspects of the present disclosure;

FIG. 6 illustrates example locations for sensory input in accordance with one or more aspects of the present disclosure;

FIG. 7 illustrates a computing device in accordance with one or more aspects of the present disclosure;

FIG. 8 illustrates a system diagram for processing and analyzing user athletic activity data for a virtual point system in accordance with one or more aspects of the present disclosure;

FIG. 9 illustrates a data flow schematic for processing and analyzing user athletic activity data for a virtual point system in accordance with one or more aspects of the present disclosure;

FIG. 10 illustrates a flowchart diagram of one or more processes for processing and analyzing user athletic activity data for a virtual point system in accordance with one or more aspects of the present disclosure;

FIG. 11 illustrates a flowchart diagram of one or more processes for setting up a user profile as part of a virtual point system in accordance with one or more aspects of the present disclosure; and

FIGS. 12-17 illustrate various user interfaces of a virtual point system in accordance with one or more aspects of the present disclosure.

### DETAILED DESCRIPTION

In the following description, reference is made to the accompanying drawings, which form a part hereof, and in which is shown by way of illustration various embodiments in which aspects of the disclosure may be practiced. It is to be understood that other embodiments may be utilized and structural and functional modifications may be made without departing from the scope and spirit of the present disclosure.

The following description is presented to enable one of ordinary skill in the art to make and use the aspects of the present disclosure. Reference is made to the accompanying drawings, which form a part hereof, and in which is shown by way of illustration, various embodiments of the present disclosure that can be practiced. It is to be understood that other embodiments can be utilized. Various modifications to the described embodiments will be apparent to those with skill in the art, and the general principles defined herein may be applied to other embodiments. Therefore, the present disclosure is not intended to be limited to the particular embodiments shown and described, but is to be accorded the widest scope consistent with the principles and novel features herein disclosed.

Certain aspects of present disclosure relate to systems, apparatuses, and methods for monitoring, analyzing, and tracking athletic activity data. Using improved processing and analytical techniques, systems described herein may efficiently process and analyze athletic activity data and provide a virtual reward system based on analyzed athletic activity data.

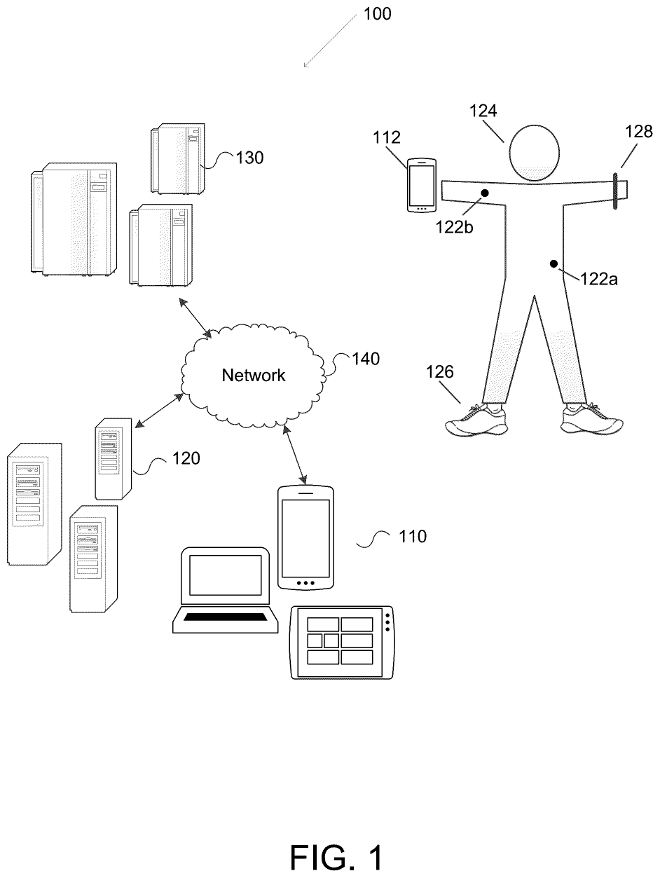

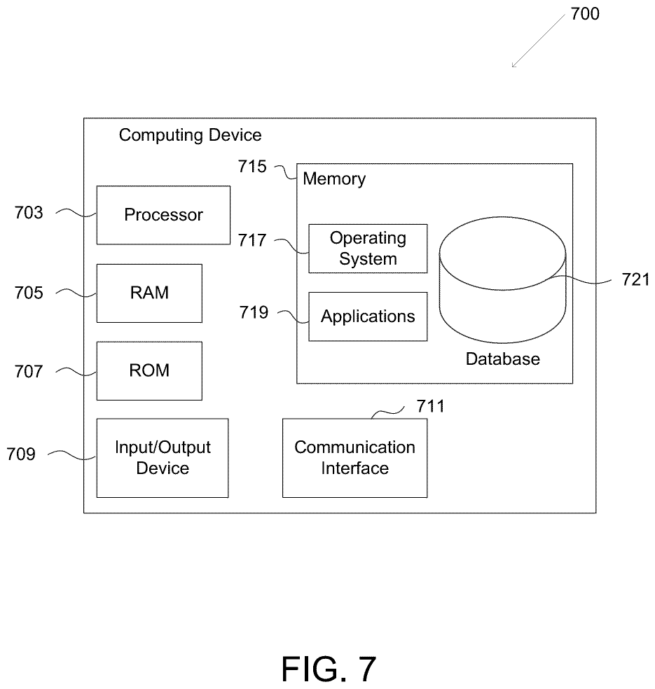

FIG. 1 illustrates an operating environment 100 in accordance with aspects of the present disclosure. The operating environment 100 may include at least one computing device 110, at least one processing server system 120, and at least one tracking server system 130, each in communication via a network 140. Any of the devices and systems described herein can be implemented, in whole or in part, using one or more computing systems described with respect to FIG. 7, described in more detail below.

Computing devices 110 may receive and display tracked athletic activity data and/or process and compile virtual athletic activity points in association with one or more user accounts as described herein. Processing server systems 120 may obtain athletic activity data, perform various verification analyses on the athletic activity data, and/or generate verification reports and/or virtual activity point updates as described in more detail herein. Tracking server systems 130 may track virtual activity points associated with one or more user profiles and track various user profile information as described herein. However, it should be noted that any of the computing devices 110, processing server systems 120, and/or tracking server systems 130 may perform some or all of any step of any process as described herein. The network 140 can include a local area network (LAN), a wide area network (WAN), a wireless telecommunications network, and/or any other communication network or combination thereof.

In some aspects, one or more computing devices 110 may be configured to sense, detect, and/or measure an athletic parameter from a user, such as user 124. Examples include, but are not limited to: an accelerometer, a gyroscope, a location-determining device (e.g., GPS), light (including non-visible light) sensor, temperature sensor (including ambient temperature and/or body temperature), sleep pattern sensors, heart rate monitor, image-capturing sensor, moisture sensor, force sensor, compass, angular rate sensor, and/or combinations thereof among others.

In some aspects, one or more computing devices 110 may receive athletic activity data files from one or more user devices as described herein, and may store such data files in association with one or more athletic parameters received from the user 124. In that regard, user 124 may be associated with (e.g., possess, carry, wear, and/or interact with) any number of devices, such as the one or more computing devices 110, shoe-mounted device 126, and/or wrist-worn device 128. One or more devices 110, 126, 128, may be specially designed for capturing athletic data which may be analyzed in conjunction with user profile information.

In certain embodiments, shoe-mounted device 126, and/or wrist-worn device 128 may be formed within or otherwise associated with user's 124 clothing or accessories, including a watch, armband, wristband, necklace, shirt, shoe, or the like. These devices may be configured to capture physical activity data of the user 124. It is to be understood that shoe-mounted device 126, and/or wrist-worn device 128 may detect data based on one or more athletic movements, e.g., in accordance with user interactions with the one or more computing devices 110 and/or operate independently of the one or more computing devices 110 (or any other device disclosed herein). For example, one or more devices may be configured to function as an all-day activity monitor that measures activity regardless of the user's proximity or interactions with the one or more computing devices 110. In certain embodiments, the shoe-mounted device 126 may comprise footwear which may include one or more sensors, including but not limited to those disclosed herein and/or known in the art.

In some embodiments, an example sensory location, such as elements 122a and 122b, may be associated with a physical apparatus, such as a sensor, data acquisition unit, or other device. Yet in some embodiments, an example sensory location may include a specific location of a body portion, region, or worn product that is monitored. In some embodiments, elements 122a and 122b may include sensors integrated into apparel, such as athletic clothing. Such sensors may be placed at any desired location of the body of user 124. Sensors 122a and 122b may communicate (e.g., wirelessly) with one or more devices (including other sensors).

As discussed herein, the data transferred to and from various devices in the operating environment 100 may include secure and sensitive data, such as confidential documents, user profiles, medical data, and/or procedures for developing documents. Therefore, it may be desirable to protect transmissions of such data using secure network protocols and encryption, and/or to protect the integrity of the data when stored on the various computing devices within the operating environment 100. For example, a file-based integration scheme or a service-based integration scheme may be used for transmitting data between the various computing devices. Data may be transmitted using various network communication protocols. Secure data transmission protocols and/or encryption can be used in file transfers to protect the integrity of the data, for example, File Transfer Protocol (FTP), Secure File Transfer Protocol (SFTP), and/or Pretty Good Privacy (PGP) encryption.

In some embodiments, one or more web services may be implemented within the various computing devices. Web services may be accessed by authorized external devices and users to support input, extraction, and manipulation of data between the various computing devices in the operating environment 100. Web services built to support a personalized display system may be employed cross-domain and/or cross-platform, and may be built for enterprise use. Such web services may be developed in accordance with various web service standards, such as the Web Service Interoperability (WS-I) guidelines. Data may be transmitted using the Secure Sockets Layer (SSL) or Transport Layer Security (TLS) protocol to provide secure connections between the computing devices. Web services may be implemented using the WS-Security standard, which provides for secure SOAP messages using XML encryption. Specialized hardware may be used to provide secure web services. For example, secure network appliances may include built-in features such as hardware-accelerated SSL and HTTPS, WS-Security, and/or firewalls. Such specialized hardware may be installed and configured in the operating environment 100 in front of one or more computing devices such that any external devices can communicate directly with the specialized hardware.

It will be appreciated that the network connections shown are illustrative and other means of establishing a communications link between the computers may be used. The existence of any of various network protocols such as TCP/IP, Ethernet, FTP, HTTP and the like, and of various wireless communication technologies such as GSM, CDMA, WiFi, and WiMAX, is presumed, and the various computing devices described herein may be configured to communicate using any of these network protocols or technologies.

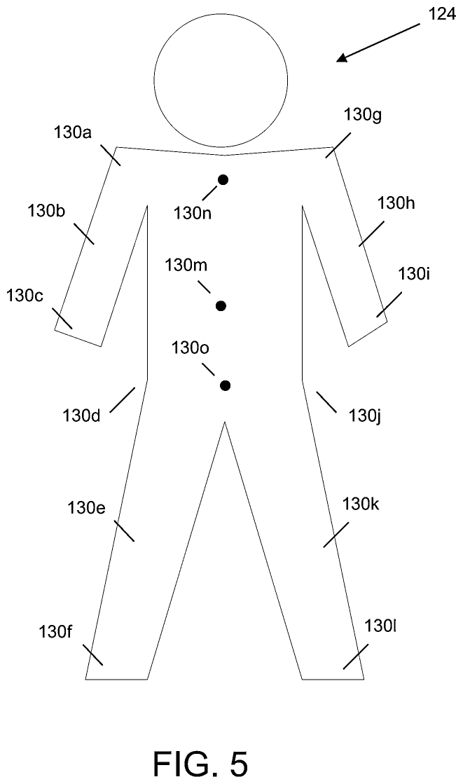

Still referring to FIG. 1, user 124 may be associated with (e.g., possess, carry, wear, and/or interact with) any number of devices, such as portable device 112, shoe-mounted device 126, wrist-worn device 128 and/or a sensing location, such as sensing location, such as locations illustrated in FIG. 5, which may comprise a physical device or a location that is used to collect information. One or more devices 112, 126, 128, and/or 130 may not be specially designed for fitness or athletic purposes. Indeed, aspects of this disclosure relate to utilizing data from a plurality of devices, some of which are not fitness devices, to collect, detect, and/or measure athletic data. In certain embodiments, one or more devices of network 140 (or any other network) may comprise a fitness or sporting device that is specifically designed for a particular sporting use. As used herein, the term “sporting device” includes any physical object that may be used or implicated during a specific sport or fitness activity. Exemplary sporting devices may include, but are not limited to: golf balls, basketballs, baseballs, soccer balls, footballs, power balls, hockey pucks, weights, bats, clubs, sticks, paddles, mats, and combinations thereof. In some embodiments, fitness devices may include objects within a sporting environment where a specific sport occurs, including the environment itself, such as a goal net, hoop, backboard, portions of a field, such as a midline, outer boundary marker, base, and combinations thereof.

In this regard, those skilled in the art will appreciate that one or more sporting devices may also be part of (or form) a structure and vice-versa, a structure may comprise one or more sporting devices or be configured to interact with a sporting device. For example, a first structure may comprise a basketball hoop and a backboard, which may be removable and replaced with a goal post. In this regard, one or more sporting devices may comprise one or more sensors, such as one or more of the sensors discussed above in relation to user 124, that may provide information utilized, either independently or in conjunction with other sensors, such as one or more sensors associated with one or more structures. For example, a backboard may comprise a first sensor configured to measure a force and a direction of the force by a basketball upon the backboard and the hoop may comprise a second sensor to detect a force. Similarly, a golf club may comprise a first sensor configured to detect grip attributes on the shaft and a second sensor configured to measure impact with a golf ball.

Portable device 112 may be a multi-purpose electronic device, that for example, includes a telephone or digital music player, including an IPOD®, IPAD®, or iPhone®, brand devices available from Apple, Inc. of Cupertino, California or Zune® or Microsoft® Windows devices available from Microsoft of Redmond, Washington. As known in the art, digital media players can serve as an output device, input device, and/or storage device for a computer. Device 112 may be configured as an input device for receiving raw or processed data collected from one or more devices in operating environment 100. In one or more embodiments, portable device 112 may include one or more components of a computer device. For example, portable device 112 may be include a display, image-capturing device, and/or one or more data acquisition devices, with or without additional components, so as to comprise a mobile terminal.

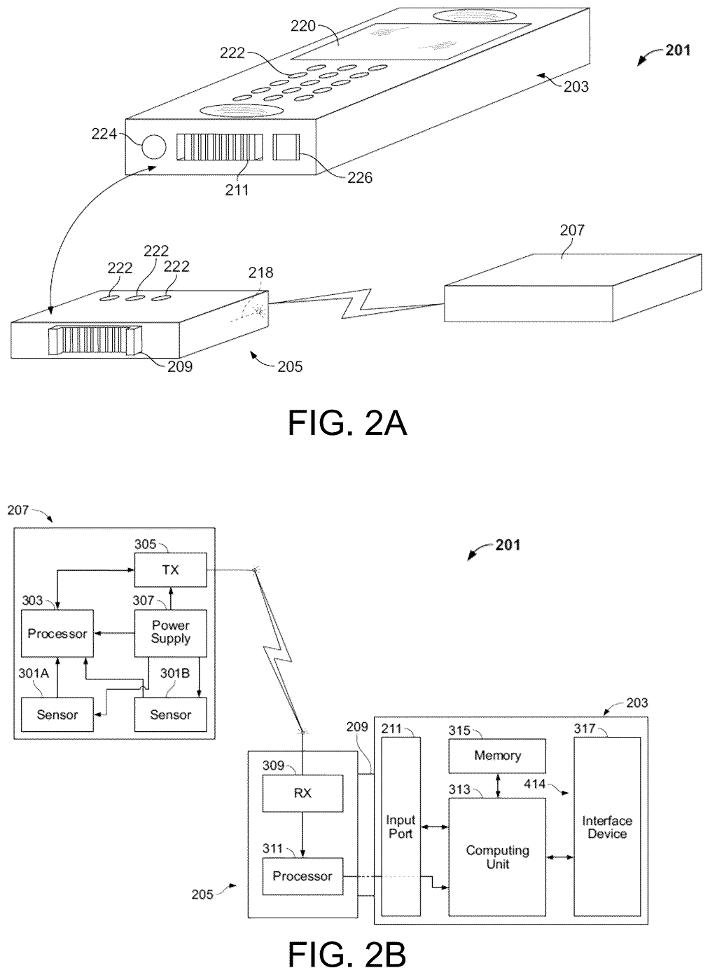

FIG. 2A illustrates one example of an athletic information monitoring device 201 that may be employed according to various examples of the present disclosure to measure athletic information corresponding a user's athletic activity. As shown in FIG. 2A, the athletic information monitoring device 201 may include a digital music player 203, an electronic interface device 205, and an athletic parameter measurement device 207. As will be described in more detail, the digital music player 203 may be (releasably) connected to the electronic interface device 205, and the combination is worn or otherwise carried by the user while he or she is performing an athletic activity, such as running or walking. The athletic parameter measurement device 207 may also be worn or carried by the user while he or she is performing an athletic activity, and may measure one or more athletic parameters relating to the athletic performance being performed by the user. The athletic parameter measurement device 207 may transmit signals to the electronic interface device 205 that correspond to the measured athletic parameter. The electronic interface device 205 may receive the signals from the athletic parameter measurement device 207 and provide the received information to the digital music player 203 (optionally, after data processing within the interface device 205).

As shown in more detail in FIG. 2A, the athletic parameter measurement device 207 may include one or more sensors 301 for measuring an athletic parameter associated with a person wearing or otherwise using the athletic parameter measurement device 207. For example, the sensors 301A and 301B may be accelerometers (such as piezoelectric accelerometers) for measuring the acceleration of the athletic parameter measurement device 207 in two orthogonal directions. The athletic parameter measurement device 207 may be carried or otherwise worn by a user to measure the desired athletic parameter while the user exercises. For example, the athletic parameter measurement device 207 may be located the sole of a user's shoe while the user walks or runs. With this arrangement, the sensors 301A and 301B may produce electrical signals corresponding to the movement of the user's foot. These signals may then be used to generate athletic data representative of the athletic activity performed by the user (e.g., the speed and/or distance moved by the user).

The athletic parameter measurement device 207 may also include a processor 303 for processing the electrical signals output by the sensors 301. The processor 303 may include a programmable microprocessor. In some examples, however, the processor 303 may be a purpose-specific circuit device, such as an ASIC. The processor 303 may perform any desired operation on the signals output from the sensors 301, such as curve smoothing, noise filtering, outlier removal, amplification, summation, integration, or the like. The processor 303 may provide the processed signals to a transmitter 305. The athletic parameter measurement device 207 may also include a power supply 307, for providing power to the sensors 301, the processor 303, and the transmitter 305 as needed. The power supply 307 may include, for example, a battery.

The athletic parameter measurement device 207 may transmit processed signals to the electronic interface device 205. The electronic interface device 205 may include a receiver 309 which receives processed signals transmitted by the transmitter 305 in the athletic parameter measurement device 207. The receiver 309 may relay the processed signals to a second processor 311, which processes the signals further. Like the processor 303, the processor 311 may perform any desired operation on the processed signals, such as curve smoothing, noise filtering, outlier removal, amplification, summation, integration, or the like. Alternatively, if desired, the interface device 205 may simply pass the signals from the transmitter 305 to the digital music player 203 without any further processing.

The processor 303 may provide processed signals to the digital music player 203. Referring back now to FIG. 2A, the electronic interface device 205 may include a connector system 209 that physically plugs into and connects with a conventional input port 211 provided on digital music player 203. The input port 211 into which the connector system 209 of the electronic interface device 205 connects may be any desired type of input port for transferring data, such as a parallel data port, a serial data port, USB port, lightning port, an earphone or microphone jack, etc. The connector system 209 may include any suitable connecting devices, such as wires, pins, electrical connectors, and the like, so as to make an electrical connection or other suitable connection with corresponding elements provided in the input port 211 of the digital music player 203 (e.g., to allow electronic and/or data communications between the interface device 205 and the digital music player 203). Additional securing elements may be provided to securely connect the interface device 205 to the digital music player 203, such as straps, hooks, buckles, clips, clamps, clasps, retaining elements, mechanical connectors, and the like.

Returning now to FIG. 2B, the processor 311 may provide the processed signals to the computing unit 313 of the digital music player 203. The computing unit 313 may initially store the processed signals in the memory 315. The computing unit 313 may operate on the processed signals provided by the athletic information monitoring device 201 to generate a “real-time” set of athletic data corresponding to the athletic activity performed by the user. For example, if the athletic information monitoring device 201 includes accelerometers for measuring the movement of the user's foot, the computing unit 313 may analyze the processed signals from the athletic information monitoring device 201 to generate a set of athletic data describing the user's speed at specific instances during the user's athletic activity and the total distance traveled by the user at each of those specific instances. Various techniques for determining a user's speed from accelerometer signals may be employed.

The athletic data set may also include a time value associated with each speed value and/or each distance value. In some examples, the athletic information monitoring device 201 may be employed to collect athletic information from different users, and the athletic data computing unit 313 may additionally prompt the user to identify himself or herself in some way. This identification information may then be included with the athletic data set generated from the information provided by the athletic information monitoring device 201, as will be discussed in more detail below. Once the computing unit 313 has generated a set of athletic data from the information provided by the athletic information monitoring device 201, the computing unit 313 may store the athletic data set in the memory 315. As will be discussed in more detail below, when the digital music player 203 is connected to a computing device implementing an athletic information collection tool, the computing unit 313 may download the athletic data to a display configuration tool hosted on a remote computing device.

While wireless communication between the athletic parameter measurement device 207 and the interface device 205 is described in the example illustrated in FIGS. 2A-2B, any desired manner of communicating between the athletic parameter measurement device 207 and the interface device 205 may be used without departing from the scope of the present disclosure, including wired connections. Also, any desired way of placing data derived from the physical or physiological data from the athletic parameter measurement device 207 in the proper form or format for display on or output from digital music player 203 may be provided without departing from the scope of the present disclosure. For example, if desired, the athletic parameter measurement device 207 may be specially designed and/or programmed for use with one or more specific electronic devices, e.g., pre-programmed and/or wired to operate with a specific device or devices and to provide output data in a form and format suitable for those devices. In this situation, the interface devices 205 may be marketed and sold to specifically target certain electronic devices, such as specific models of digital music players and even other electronic devices, such as telephones, watches, personal digital assistants, and the like. As another option, if desired, the interface devices 205 may be programmed at a later time to operate with a wide variety of different electronic devices, e.g., by downloading display or device driver and/or format data for specific electronic devices from the Internet, from disk, from another source, and the like.

In accordance with one or more aspects of the present disclosure, the electronic interface device 205 and/or digital music player 203 may further include a display 220 and/or a user input system 222, such as one or more rotary input devices, switches, buttons (as shown in the illustrated example in FIG. 2A), mouse or trackball elements, touch screens, or the like, or some combination thereof. The display 220 may be employed to show, for example, information relating to music being played by the digital music player 203, information relating to the athletic information signals being received by the digital music player 203, athletic data being generated by the digital music player 203 from the received athletic information signals, etc. The user input system 222 may be employed, for example: to control one or more aspects of the processing of the input data received via interface device 205, to control input data receipt (e.g., timing, types of information received, on-demand data requests, etc.), to control data output to or by the digital music player 203, to control the athletic parameter measurement device 207, etc. Alternatively or additionally, if desired, the input system on the digital music player 203 (e.g., buttons 222, a touch screen, a digitizer/stylus based input, a rotary input device, a trackball or roller ball, a mouse, etc.), may be used to provide user input data to the interface device 205 and/or to the athletic parameter measurement device 207. As still another example, if desired, a voice input system may be provided with the interface device 205 and/or the digital music player 203, e.g., to enable user input via voice commands. Any other desired type of user input system, for control of any system elements and/or for any purpose, may be provided without departing from the scope of the present disclosure.

The digital music player 203 may include additional input and/or output elements, e.g., such as ports 224 and 226 shown in FIG. 2A, e.g., for headphones (or other audio output), power supplies, wireless communications, infrared input, microphone input, or other devices. If desired, and if these ports 224 and/or 226 would be covered when the interface device 205 is attached to the digital music player 203, the interface device 205 may be equipped with similar external ports to ports 224 and/or 226, and internal circuitry may be provided in the interface device 205 to enable the user to plug the same additional devices into the interface device 205 as they might plug into the digital music player 203 and still take advantage of the same functions (e.g., to thereby allow the necessary data, signals, power, and/or information to pass through the interface device 205 to the user, to another output, and/or to the digital music player 203).

It should be appreciated that, while some specific examples described herein above relate to a digital music player 203, alternate or additional examples may be implemented using any portable electronic device. For example, the athletic parameter measurement device 207 may be used in conjunction with a mobile telephone, a watch, a personal digital assistant, another type of music player (such as a compact disc or satellite radio music player), a portable computer, or any other desired electronic device. Still further, some examples may alternately or additionally omit the use of the interface device 205. For example, the athletic parameter measurement device 207 may be configured to communicate using the Bluetooth wireless communication protocol, so that it can be employed with Bluetooth-capable mobile telephones, personal digital assistants, watches or personal computers. Of course, still other wireless or wired communication techniques may be employed while omitting the interface device 205.

It also should be appreciated that, while a specific example of an athletic parameter measurement device 207 has been described above for ease of understanding, any type of desired athletic parameter measurement device 207 may be employed without departing from the scope of the present disclosure. For example, in some implementations, the athletic parameter measurement device 207 may be a heart rate monitor, a blood oxygen monitor, a satellite positioning device (e.g., a Global Positioning Satellite (GPS) navigation device), a device for measuring the electrical activity of the user (e.g., an EKG monitor), or any other device that measures one or more physical parameters of the user. Still further, the athletic parameter measurement device 207 may measure one or more operational parameters of some device being manipulated by the user, such as the speed and/or distance moved on a bicycle; the speed and/or work performed using a treadmill, rowing machine, elliptical machine, stationary bicycle, or the like; and the speed and/or distance traveled using skis (water or snow), skates (roller or ice), or snowshoes or the like worn by the user, and the like.

Also, while the athletic parameter measurement device 207 has been described as being separate from the digital music player 203 or other portable electronic device that receives the signals from the athletic parameter measurement device 207, with some implementations, the athletic parameter measurement device 207 may be incorporated into the digital music player 203 or other portable electronic device. For example, some implementations may employ a music player, mobile telephone, watch or personal digital assistant that incorporates accelerometers, a satellite positioning device, or any other desired device for measuring athletic activity. Still further, it should be appreciated that various implementations may employ a plurality of athletic parameter measurement devices 207, incorporated into the digital music player 203 or other portable electronic device, separate from the digital music player 203 or other portable electronic device, or some combination thereof.

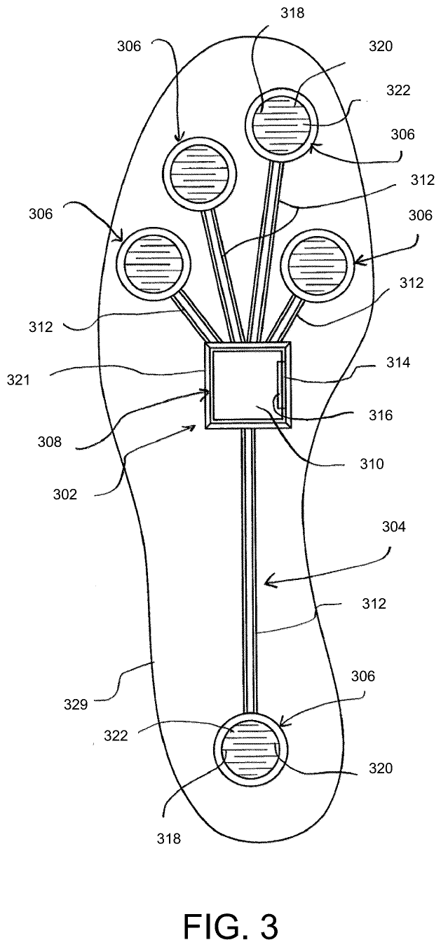

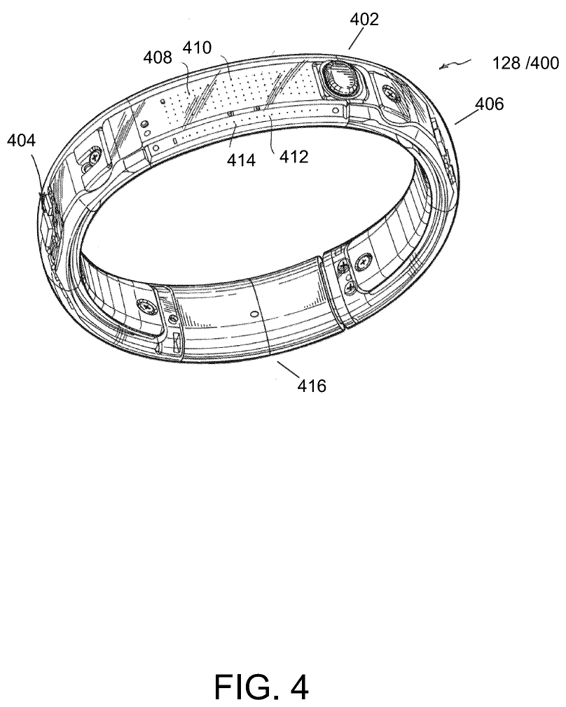

Referring back to FIG. 1, in certain embodiments, I/O devices may be formed within or otherwise associated with user's 124 clothing or accessories, including a watch, armband, wristband, necklace, shirt, shoe, or the like. These devices may be configured to monitor athletic movements of a user. It is to be understood that they may detect athletic movement during user's 124 interactions with device 112 and/or operate independently of device 112 (or any other device disclosed herein). For example, one or more devices in network 102 may be configured to function as an all-day activity monitor that measures activity regardless of the user's proximity or interactions with device 112. It is to be further understood that the sensory system 302 shown in FIG. 3 and the device assembly 400 shown in FIG. 4, each of which are described in the following paragraphs, are merely illustrative examples.

### Shoe-Mounted Device

In certain embodiments, device 126 shown in FIG. 1, may include footwear which may include one or more sensors, including but not limited to those disclosed herein. FIG. 3 illustrates one example embodiment of a sensor system 302 providing one or more sensor assemblies 304. Assembly 304 may include one or more sensors, such as for example, an accelerometer, gyroscope, location-determining components, force sensors and/or or any other sensor disclosed herein or known in the art. In the illustrated example, assembly 304 incorporates a plurality of sensors, which may include force-sensitive resistor (FSR) sensors 306; however, other sensor(s) may be utilized. Port 308 may be positioned within a sole structure 329 of a shoe, and is generally configured for communication with one or more electronic devices. Port 308 may optionally be provided to be in communication with an electronic module 310, and the sole structure 329 may optionally include a housing 321 or other structure to receive the module 310. The sensor system 302 may also include a plurality of leads 312 connecting the FSR sensors 306 to the port 308, to enable communication with the module 310 and/or another electronic device through the port 308. Module 310 may be contained within a well or cavity in a sole structure of a shoe, and the housing 321 may be positioned within the well or cavity. In some examples, at least one gyroscope and at least one accelerometer may be provided within a single housing, such as module 310 and/or housing 321. In some examples, one or more sensors may be provided that, when operational, are configured to provide directional information and angular rate data. The port 308 and the module 310 may include complementary interfaces 314, 316 for connection and communication.

In certain examples, at least one force-sensitive resistor 306 shown in FIG. 3 may contain first and second electrodes or electrical contacts 318, 320 and a force-sensitive material 322 disposed between the electrodes 318, 320 to electrically connect the electrodes 318, 320 together. When pressure is applied to the force-sensitive material 322, the resistivity and/or conductivity of the force-sensitive material 322 changes, which changes the electrical potential between the electrodes 318, 320. The change in resistance can be detected by the sensor system 302 to detect the force applied on the FSR sensors 306. The force-sensitive material 322 may change its resistance under pressure in a variety of ways. For example, the force-sensitive material 322 may have an internal resistance that decreases when the material is compressed. Further embodiments may utilize “volume-based resistance”, which may be implemented through “smart materials.” As another example, the material 322 may change the resistance by changing the degree of surface-to-surface contact, such as between two pieces of the force sensitive material 322 or between the force sensitive material 322 and one or both electrodes 318, 320. In some circumstances, this type of force-sensitive resistive behavior may be described as “contact-based resistance.”

### Wrist-Worn Device

As shown in FIG. 4, device 400 (which may resemble or comprise sensory device 128 shown in FIG. 1), may be configured to be worn by user 124, such as around a wrist, arm, ankle, neck or the like. Device 400 may include an input mechanism, such as a depressible input button 402 configured to be used during operation of the device 400. The input button 402 may be operably connected to a controller 404 and/or any other electronic components, such as one or more of the elements discussed in relation to device 112 shown in FIG. 1. Controller 404 may be embedded or otherwise part of housing 406. Housing 406 may be formed of one or more materials, including elastomeric components and comprise one or more displays, such as display 408. The display may be considered an illuminable portion of the device 400. The display 408 may include a series of individual lighting elements or light members such as LED lights 410. The lights may be formed in an array and operably connected to the controller 404. Device 400 may include an indicator system 412, which may also be considered a portion or component of the overall display 408. Indicator system 412 may operate and illuminate in conjunction with the display 408 (which may have pixel member 414) or completely separate from the display 408. The indicator system 412 may also include a plurality of additional lighting elements or light members, which may also take the form of LED lights in one or more aspects. In certain examples, indicator system may provide a visual indication of goals, such as by illuminating a portion of lighting members of indicator system 412 to represent accomplishment towards one or more goals. Device 400 may be configured to display data expressed in terms of activity points or currency earned by the user based on the activity of the user, either through display 408 and/or indicator system 412.

A fastening mechanism 416 may be disengaged s the such that device 400 can be positioned around a wrist or portion of the user 124 and the fastening mechanism 416 can be subsequently placed in an engaged position. In some examples, fastening mechanism 416 may include an interface, including but not limited to a USB port, for operative interaction with computer device 114 and/or devices, such as devices 110 and/or 112. In certain examples, fastening member may include one or more magnets. In one example, fastening member may be devoid of moving parts and rely entirely on magnetic forces.

In certain examples, device 400 may include a sensor assembly (not shown in FIG. 4). The sensor assembly may include a plurality of different sensors, including those disclosed herein and/or known in the art. In one example, the sensor assembly may include or permit operative connection to any sensor disclosed herein or known in the art. Device 400 and or its sensor assembly may be configured to receive data obtained from one or more external sensors.

Elements 122a and 122b of FIG. 1 show example sensory locations which may be associated with a physical apparatus, such as a sensor, data acquisition unit, or other device. In certain examples, elements 122a and 122b may each include a sensor, such that elements 122a and 122b may include sensors integrated into apparel, such as athletic clothing/athletic apparel. Such sensors may be placed at any desired location of the body of user 124. Sensors 122a and 122b may communicate (e.g., wirelessly) with one or more devices (including other sensors) over network 140. In certain examples, passive sensing surfaces may reflect waveforms, such as infrared light, emitted by image-capturing device on device 110. In one example, passive sensors located on user's 124 apparel may include generally spherical structures made of glass or other transparent or translucent surfaces which may reflect waveforms. Different classes of apparel may be utilized in which a given class of apparel has specific sensors configured to be located proximate to a specific portion of the user's 124 body when properly worn. For example, golf apparel may include one or more sensors positioned on the apparel in a first configuration and yet soccer apparel may include one or more sensors positioned on apparel in a second configuration.

FIG. 5 shows illustrative locations for sensory input (see, e.g., sensory locations 130a-1300). In this regard, sensors may be physical sensors located on/in a user's clothing, yet in other embodiments, sensor locations 130a-130o may be based upon identification of relationships between two moving body parts. For example, sensor location 130a may be determined by identifying motions of user 124 with an image-capturing device, such as image-capturing device 118. Thus, in certain embodiments, a sensor may not physically be located at a specific location (such as one or more of sensor locations 130a-1300), but is configured to sense properties of that location, such as with an image-capturing device or other sensor data gathered from other locations. In this regard, the overall shape or portion of a user's body may permit identification of certain body parts. Regardless of whether an image-capturing device is utilized and/or a physical sensor located on the user 124, and/or using data from other devices, (such as sensory system 302), device assembly 400 and/or any other device or sensor disclosed herein or known in the art is utilized, the sensors may sense a current location of a body part and/or track movement of the body part. In one embodiment, sensory data relating to location 130m may be utilized in a determination of the user's center of gravity (center of mass). For example, relationships between location 130a and location(s) 130f/130l with respect to one or more of location(s) 130m-130o may be utilized to determine if a user's center of gravity has been elevated along the vertical axis (such as during a jump) or if a user is attempting to “fake” a jump by bending and flexing their knees. In one embodiment, sensor location 1306n may be located at about the sternum of user 124. Likewise, sensor location 130o may be located approximate to the naval of user 124. In certain embodiments, data from sensor locations 130m-130o may be utilized (alone or in combination with other data) to determine the center of gravity for user 124. In further embodiments, relationships between multiple sensor locations, such as sensors 130m-130o, may be utilized in determining orientation of the user 124 and/or rotational forces, such as twisting of user's 124 torso. Further, one or more locations, such as location(s), may be utilized as (or approximate) a center of moment location. For example, in one embodiment, one or more of location(s) 130m-130o may serve as a point for a center of moment location of user 124. In another embodiment, one or more locations may serve as a center of moment of specific body parts or regions.

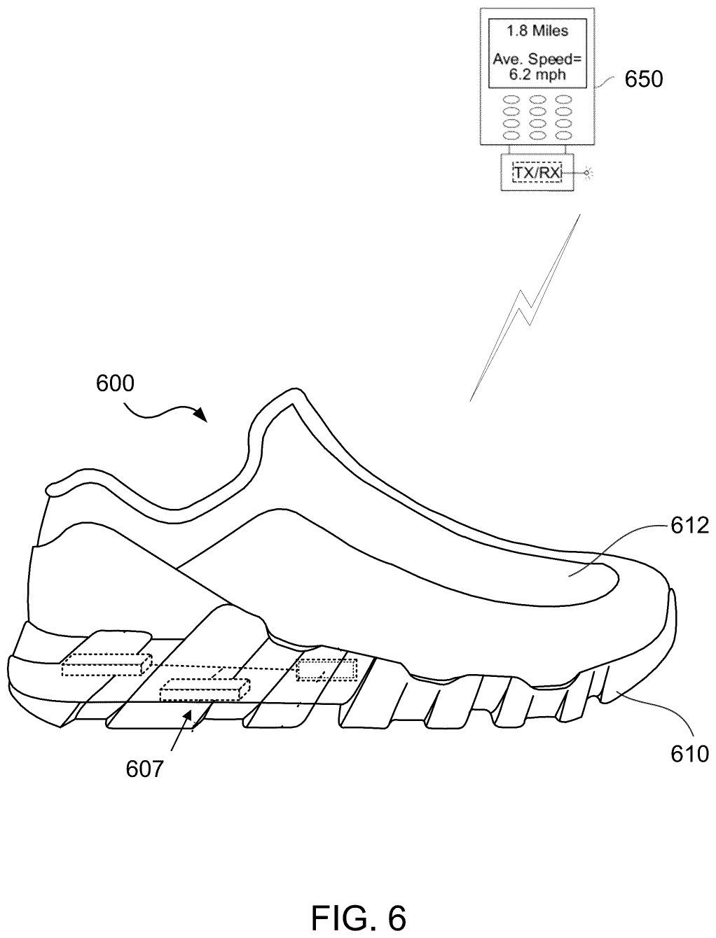

FIG. 6 schematically depicts an article of footwear according to example embodiments disclosed herein. The article of footwear 600 may include any type of footwear configured to be worn while participating in an athletic activity or for other casual or formal occasions. The article of footwear 600 may be referred to as a shoe 600, and may not be limited to enclosed footwear embodiments. As shown in FIG. 6, the athletic parameter measurement device 607 may be located the sole of a user's shoe 600 while the user walks or runs. With this arrangement, the sensors 301A, 301B will produce electrical signals corresponding to the movement of the user's foot. These signals may then be used to generate athletic data representative of the athletic activity performed by the user (e.g., the speed and/or distance moved by the user).

It is contemplated that the relative positioning of the athletic parameter measurement device 607 may differ to that schematically depicted in FIG. 6. In one example, athletic parameter measurement device 607 may be encapsulated within a sole structure 610 of the article of footwear 600. In alternative implementations, athletic parameter measurement device 607 may be positioned within one or more structures of an upper 612 of the article of footwear 600. In one example, athletic parameter measurement device 607 may be configured with an interface for receiving electrical energy from an external source. This interface may include a connection port configured to receive a wired connection, or may be an interface configured for wireless charging when the article of footwear 600 is placed in proximity with an external wireless charging dock or source. In addition, the athletic parameter measurement device 607 may be configured to receive data from one or more sources external to the article of footwear 600. As such, the athletic parameter measurement device 607 may include an interface configured to receive and transmit wired or wireless data from and to an external data source, such an external computing device 650. This external computing device 650 may be configured to receive activity data from the athletic parameter measurement device 607, or may be configured to update firmware on \ the athletic parameter measurement device 607, among other functions. It is contemplated that the athletic parameter measurement device 607 may be configured to be encapsulated within the article of footwear 600 such that each element is shielded from contaminants such as dust, dirt, or water, among others.

Turning now to FIG. 7, a computing device 700 in accordance with one or more aspects of the present disclosure is shown. The computing device 700 may include a processor 703 for controlling overall operation of the computing device 700 and its associated components, including RAM 705, ROM 707, input/output device 709, communication interface 711, and/or memory 715. A data bus may interconnect processor(s) 703, RAM 705, ROM 707, memory 715, I/O device 709, and/or communication interface 711. Communication interface 711 may include one or more transceivers, digital signal processors, and/or additional circuitry and software for communicating via any network, wired or wireless, using any protocol including those described herein.

Input/output (I/O) device 709 may include a microphone, keypad, touch screen, and/or stylus through which a user of the computing device 700 may provide input, and may also include one or more of a speaker for providing audio output and a video display device for providing textual, audiovisual, and/or graphical output. In some embodiments, the I/O devices 709 may include one or more sensors and/or one or more image capture devices. The image capture devices may be used to capture images of a subject. The sensors may be used to determine viscoelastic tissue parameters in images captured using one or more image capture devices. For example, I/O device 709 may include an ultrasound shear wave elastography apparatus. Software may be stored within memory 715 to provide instructions to processor 703 allowing computing device 700 to perform various actions. For example, memory 715 may store software used by the computing device 700, such as an operating system 717, application programs 719, and/or an associated internal database 721. The various hardware memory units in memory 715 may include volatile and nonvolatile, removable and non-removable media implemented in any method or technology for storage of information such as computer-readable instructions, data structures, program modules, or other data. Memory 715 may include one or more physical persistent memory devices and/or one or more non-persistent memory devices. Memory 715 may include, but is not limited to, random access memory (RAM) 705, read only memory (ROM) 707, electronically erasable programmable read only memory (EEPROM), flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by processor 703.

Processor 703 may include a single central processing unit (CPU), which may be a single-core or multi-core processor (e.g., dual-core, quad-core, etc.), or may include multiple CPUs. Processor(s) 703 and associated components may allow the computing device 700 to execute a series of computer-readable instructions to perform some or all of the processes described herein. Although not shown in FIG. 7, various elements within memory 715 or other components in computing device 700, may include one or more caches, for example, CPU caches used by the processor 703, page caches used by the operating system 717, disk caches of a hard drive, and/or database caches used to cache content from database 721. For embodiments including a CPU cache, the CPU cache may be used by one or more processors 703 to reduce memory latency and access time. A processor 703 may retrieve data from or write data to the CPU cache rather than reading/writing to memory 715, which may improve the speed of these operations. In some examples, a database cache may be created in which certain data from a database 721 is cached in a separate smaller database in a memory separate from the database, such as in RAM 705 or on a separate computing device. For instance, in a multi-tiered application, a database cache on an application server may reduce data retrieval and data manipulation time by not needing to communicate over a network with a back-end database server. These types of caches and others may be included in various embodiments, and may provide potential advantages in certain implementations of document development systems, such as faster response times and less dependence on network conditions when transmitting and receiving data.

Although various components of computing device 700 are described separately, functionality of the various components may be combined and/or performed by a single component and/or multiple computing devices in communication without departing from the scope of the present disclosure.

### Data Collection and Display Tools

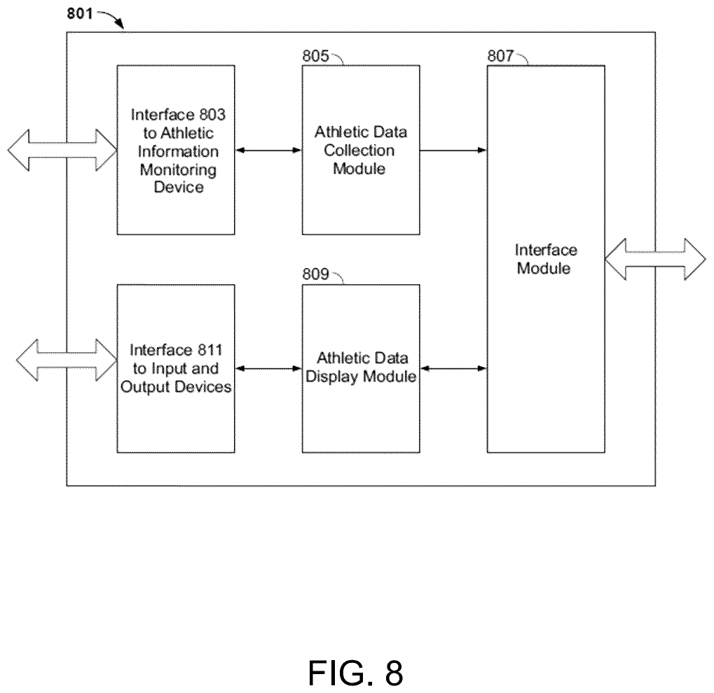

FIG. 8 illustrates an example of an athletic information collection and display device 801 that may be employed to collect and/or display athletic data according to various aspects of the present disclosure. As will be discussed in more detail below, the athletic information collection and display device 801 may both collect and display athletic data. The athletic information collection and display device 801 may be implemented using any suitable variation of the computing device 110 or device 112 previously described. In some situations, however, the information collection and display device 801 may be commercially implemented using a desktop or laptop personal computer using, e.g., a version of the Microsoft Windows operating system available from Microsoft Corporation of Redmond, Washington, a version of the Apple Macintosh operating system available for Apple Corporation of Cupertino, California, or a version of the Unix or Linux operating systems available from a plurality of vendors.

As shown FIG. 8, the athletic information collection and display device 801 includes an interface 803 for receiving data from the athletic information monitoring device 201. The interface 803 may be implemented using, e.g., electrical components, software components (such as application program interfaces (APIs)), or some combination thereof. The athletic information collection and display device 801 also has an athletic data collection module 805. With various examples, the athletic data collection module 805 may detect when the digital music player 203 or other portable electronic device storing one or more athletic data sets is connected to the athletic information collection and display device 801 through the interface 803 and establish a communication session with the digital music player 203 or other portable electronic device to retrieve the athletic data set or sets. In some implementations of the present disclosure, the athletic data collection module 805 may delete athletic data sets from the digital music player 203 or other portable electronic device after the athletic data sets have been retrieved and stored at device 801.

With some examples, the athletic data collection module 805 may perform some further operations on the athletic data sets retrieved from the digital music player 203 or other portable electronic device. For example, if the athletic information monitoring device 201 can be employed to collect athletic information from different users, then the athletic data collection module 805 may additionally prompt the user to identify himself or herself (if this information was not previously obtained by the athletic information collection and display device 801). This identification information may then be included with the retrieved athletic data sets.

As previously noted, the athletic information collection and display device 801 typically will generate sets of athletic data from information measured by one or more athletic parameter measurement devices 207. With some embodiments, however, the athletic information collection and display device 801 may instead store the raw information provided by the athletic parameter measurement devices 207. With these embodiments, the athletic data collection module 805 may retrieve the raw information from the digital music player 203 or other portable electronic device, and then generate athletic data sets from the raw information itself. Still other examples may divide functions relating to the generation of athletic data from the raw information measured by athletic parameter measurement devices 207 between the athletic data collection module 805 and the digital music player 203 or other portable electronic device as desired.

The athletic data collection module 805 may be implemented by, for example, software instructions executed by a computing device 110. With some examples, the athletic data collection module 805 may be implemented by a conventional software tool, such as a browser. Alternately, athletic data collection module 805 may be implemented by a purpose-specific software tool or by a conventional software tool enhanced to perform athletic data collection functions. For example, the athletic data collection module 805 may be implemented by a software tool that incorporates a conventional browser to perform a variety of functions. These functions may include, e.g., selecting, purchasing, and downloading music and video content in addition to collecting athletic data from a digital music player 203 or other portable electronic device.

Once the athletic data collection module 805 has collected the processed signals provided by the athletic information monitoring device 201, the athletic data collection module 805 transmits the athletic data set to an athletic data display configuration device 901 through an interface module 807. The athletic information collection and display device 801 may communicate with the athletic data display configuration device 901 through a conventional network, such as the Internet. With these configurations, the interface module 807 may be implemented using any conventional type of network interface, such as a network interface card. Of course, any type of desired hardware or software combination alternately may be used to allow the athletic data collection module 805 to send the collected athletic data to the athletic data display configuration device 901. With some implementations, the athletic data collection module 805 may automatically forward collected athletic data to the athletic data display configuration device 901. For example, the athletic data collection module 805 may attempt to forward collected athletic data to the athletic data display configuration device 901 immediately after collection, at a prescheduled interval, upon the detection of a network connection to the athletic data display configuration device 901, or some combination thereof. Alternately or additionally, the athletic data collection module 805 may prompt a user to specify when collected athletic data should be sent to the athletic data display configuration device 901.

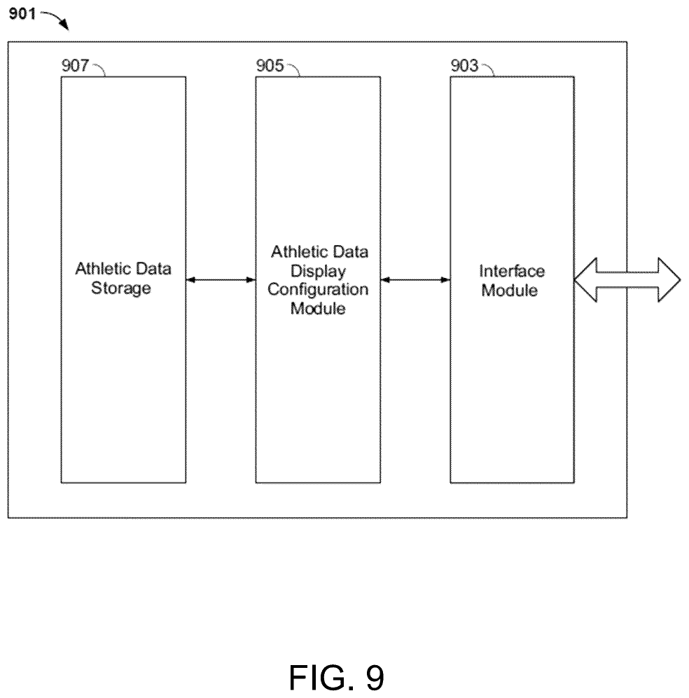

FIG. 9 illustrates an example of an athletic data display configuration device 901 that may be employed according to various aspects of the present disclosure. As seen in this figure, the athletic data display configuration device 901 includes an interface module 903 for communicating with the athletic information collection and display device 801. As previously noted, the athletic information collection and display device 801 may communicate with the athletic data display configuration device 901 through a conventional network, such as the Internet. With these configurations, the interface module 903 may be implemented using any conventional type of network interface, such as a network interface card. Of course, any type of desired hardware or software combination alternately may be used to allow the athletic data display configuration device 901 to communicate with the athletic information collection and display device 801.

The athletic data display configuration device 901 also includes an athletic data display configuration module 905, and an athletic data storage 907. When the interface 903 of the athletic data display configuration device 901 receives athletic data from the athletic information collection and display device 801, it provides the received athletic data to the athletic data display configuration module 905. The athletic data display configuration module 905 may then store the athletic data in the athletic data storage 907 for future use. As will be discussed in more detail below, the athletic data display configuration module 905 also will retrieve athletic data from the athletic data storage 907, and configure the retrieved athletic data for display through one or more user interfaces in a manner that is meaningful to a user.

Returning now to FIG. 8, when a user wishes to view information relating to his or her athletic activities (or the athletic activities of another, as will be discussed in more detail below), the user submits this request to the athletic information collection and display device 801. More particularly, the user can employ conventional input and output devices, such as a keyboard, mouse, display and the like. The display request is then provided to an athletic data display module 809 through a conventional interface input/output interface 811. The interface input/output interface 811 may be implemented using any desired combination of hardware and software components, such as conventional application programming interfaces (APIs) used to detect and process input from input devices and to send data to and otherwise control output devices.

With some examples, the athletic data display module 809 may be implemented using any conventional tool for receiving input to request and control the display of data, and then subsequently displaying the data in the manner requested. For example, the athletic data display module 809 may be implemented using a conventional browser program, such as Microsoft Internet Explorer, Mozilla Firefox, or Opera executing on a computing device 110. With still other embodiments, the athletic data display module 809 may be implemented using a conventional browser program that has been enhanced by one or more display tools, such as an ActiveX plug-in, a Java script or a version of the Macromedia Flash Player or Adobe Flash Player, available from Adobe Systems Incorporated of San Jose, California. In still other embodiments, the athletic data display module 809 may be implemented, for example, by a purpose-specific software tool for displaying athletic data.

As will be discussed in more detail below, when a user activates the athletic data display module 809, he or she is provided with a user interface prompting the user to select what collected athletic data he or she wishes to view, the format in which the user wishes to view the collected athletic data, etc. This user interface may be generated by the athletic data display module 809, the athletic data display configuration module 905, or some combination thereof. When a user employs the provided user interface to submit a request to view athletic data, the athletic data display module 809 relays the request to the athletic data display configuration module 905. In response, the athletic data display configuration module 905 configures the requested athletic data for display by the athletic data display module 809. For example, as will be discussed in more detail below, a user may request to view the total distance run by a user for each day in a one week period. In response, the athletic data display configuration module 905 will retrieve the relevant distance data from the athletic data storage 907. It will then configure the retrieved distance data to be displayed through a desired image (e.g., a bar graph) and provide the configured athletic data to the athletic data display module 809 for display to the user.

It should be noted that, with some embodiments, the data display configuration functions may be divided between the athletic data display module 809 and the athletic data display configuration module 905. For example, if the athletic data display module 809 is implemented by a simple browser, then the athletic data display module 809 may serve as a “thin client” for the athletic data display configuration module 905. That is, all of the data display configuration functions may be performed by the athletic data display configuration module 905. The athletic data display module 809 will then only display the information provided to it. Alternatively, if the athletic data display module 809 is implemented by a purpose-specific software tool, then most or all of the data display configuration functions may be performed by the athletic data display module 809. With these examples, the athletic data display configuration module 905 may be used only to store and retrieve athletic data from the athletic data storage 907.

### Activity Data Tracking, Conversion, and Storage

Wearable and non-wearable mobile devices such as athletic information monitoring device 201 (FIG. 2) and device 400 (FIG. 4) may be configured to measure athletic performance information for a variety of athletic activity types and to determine an amount of activity points earned through those activity types. For example, athletic information monitoring device 201 may include a pedometer to measure a number of steps taken during walking or running exercises and an accelerometer to measure speed during such workouts. In another example, and as discussed above, a device such as device 400 may connect (either wirelessly or through a wire) to various wearable and non-wearable sensors such as heart-rate sensors and accelerometers. The athletic measurements recorded for each of the multiple types of activity may be converted into activity points and summed to allow a user to measure his or her athletic performance across multiple types of activities. The mobile devices may store conversion rates for each type of athletic activity. If a conversion rate for a particular type of activity is not known, a default conversion rate may be used instead. Mobile devices may comprise consumer electronic devices such as watches, athletic bands, location determination devices, digital music players and the like.

Athletic performance monitoring devices may be configured specifically for one or more types of athletic activity or may provide generic tracking and monitoring for a variety of different athletic activity types. In the latter instance, one example of a generic performance monitoring device may be one that converts all movement to a number of steps taken (e.g., using a pedometer). Accordingly, regardless of whether the user is running, playing soccer, playing tennis or jumping rope, the athletic activity measure may register in steps. Accordingly, the number of athletic activity points earned through the use of such a generic monitoring device may use a step-based conversion rate. In another example, an athletic performance monitoring device may be configured to detect measures of athletic activity that are specific to a particular sport or athletic activity. For example, a monitoring device specifically configured for running may include sensors and/or software that provide running pace detection at which a user is running as well as a distance run. The running specific monitoring device may further be configured to use pace and distance information in determining a number of activity points corresponding to a running workout. In another example, a monitoring device may be configured to monitor and track soccer-related activities. Accordingly, the monitoring device may include sensors for detecting contact between a user and a ball and/or a force of impact between a user's foot and the ball for example. A conversion algorithm may then be used that incorporates such soccer-specific information in determining a number of activity points earned in a soccer workout. Other types of sports of athletic activities such as basketball, football, cycling, using gym equipment and the like may also have activity-specific measures. In addition to activity specific components and/or detection algorithms, athletic performance monitoring devices may also include generic detection systems so that the devices may be used in a wide variety of activities. For example, each of the above soccer athletic performance monitoring device and running performance monitoring device may include a general pedometer for detecting activity during non-soccer and non-running type activities, respectively.

In some examples, a relative amount of effort by the user performing an activity may be determined and used as a basis for a particular conversion rate in converting activity data to virtual points. In such examples, the number of points awarded for physical activity may be based, at least in part, on a relative amount of effort by the user in performing the physical activity. In that regard, more effort expended by the user (e.g., by running faster and/or for a longer distance, by lifting a higher weight and/or performing more repetitions, by playing a soccer game for a longer duration and/or having a higher number of steps, and the like) may translate to a larger number of points awarded than less effort expended by a user in performing that activity. Accordingly, such systems provide additional motivation for user to expend greater effort while performing an athletic activity.

In certain examples, a user baseline effort may be determined upon the user setting up a user profile, providing user information, and/or performing a baseline activity. Subsequently, upon a user performing an athletic activity, a relative amount of effort may be determined based on comparing an effort during the athletic activity with a baseline effort of the user. The baseline effort for the user may be adjusted dynamically over time as a user progresses in an athletic training program, or as a user's fitness changes over time and/or based on frequency (or infrequency) of workouts. In certain examples, a machine learning engine may be trained to dynamically determine a baseline effort level of a particular user based on receiving and analyzing ongoing activity data and determining a baseline effort level for a particular type of activity based on the received data. In some examples, user effort may be determined by comparing one or more components activity data to one or more threshold levels. In some examples, user effort may be determined based on a user achieving one or more preset conditions or goals during a workout, e.g., running a certain distance, maintaining or exceeding a certain pace, completing a preset number of laps, maintaining a heartrate within a specified range, and the like. As another example, a baseline effort may be set by attributing the user to a certain athletic categorization where each categorization may be associated with a particular baseline effort, e.g., for running a user may select from new runner, intermediate runner, long distance runner, and the like. In such examples, an indication of an approximation of user effort may be displayed to the user during and/or after an athletic activity. By using an effort level of the user as part of awarding virtual activity points, users may be further incentivized or motivated to expend more effort during a workout, thus providing enhanced benefits from a physical activity.

Devices might also be configured to be sport-specific by defining a subset of athletic activity measures that may be monitored. For example, in a football configuration, the device may be configured to only measure heart rate and pace. In another example where the monitoring device is configured for soccer, the device might be configured to only monitor speed and foot-ball impact force. Additionally, or alternatively, the type and content of feedback including motivational messages and coaching provided to the user may be sport-specific or selected based on the sport for which the monitoring device is configured. Accordingly, different messages may be provided to users depending on the sport for which the device is configured.

Moreover, by configuring a device for a particular sport, a trainer or coach may be able to push information to users based on sport. Accordingly, a trainer may develop or provide workouts that are intended for athletes in a particular sport. In such instances, the trainer may transmit the workout data to only those devices that are configured for the relevant sport or sports. In another example, a coach may transmit training profiles to each of the members of a team for a specific sport. Similarly, challenges, goals and other objectives may also be defined and transmitted in a sport-specific manner. In some arrangements, a device configured for a particular sport may ignore or discard data that is not intended for the particular sport.

Alternatively, or additionally, devices may be classified into multiple categories such as certified and non-certified devices. These classifications may be defined by an entity or organization that provides user athletic activity performance monitoring service and/or that sponsors activity point rewards. The classifications may further affect the amount or grade of activity points earned. For example, data recorded by certified devices may be treated differently from data recorded by non-certified devices. As a more specific example, activity points earned through certified devices may be considered more valuable or reliable than activity points earned through manual entry or non-certified devices. Accordingly, a user may earn 1.5 points for every 10 minutes run when measured by a certified device in contrast to 1 point for every 10 minutes run for workouts recorded using a non-certified device. Device certification may be performed by one or more authorized entities, such as an entity that provides the athletic activity point currency and access to the products and/or services that may be exchanged therewith.

Certification may include insuring that the devices include adequate security components or software for preventing the falsification of athletic activity information (e.g., calories burned, miles run, steps taken) and corresponding activity points. According to one or more aspects, certification may be enforced by storing model and/or serial numbers of certified devices in a certification database. When a system wishes to determine whether a device is a certified device, the system may query the certification database with a model or serial number of the device. Alternatively, or additionally, certified devices may include specific hardware and/or software components that are recognizable to another system or device. For example, an activity point conversion program running on a mobile device may be digitally signed by a certifying authority. Accordingly, when a user wishes to transmit or receive data to or from another device, the digital signature of the certifying authority may be transmitted to the other device for verification of certification.

In one or more arrangements, software, algorithms, product labels and/or services may be licensed to other companies for the creation of certified devices, software and/or hardware. Accordingly, third party companies or developers may create applications or add-on hardware that utilizes the activity performance monitoring features provided by an athletic activity performance monitoring and tracking service. For example, a third party developer may use application protocol interfaces (APIs) of a device to access activity tracking capabilities and information. The information may then be incorporated into third party software, such as a video game in which progression is measured by the user's athletic performance. In another example, a developer may create a mobile application that helps select music based on a current pace at which a user is running or walking, a number of steps taken, a number of calories burned, a rate at which a user is burning calories and the like. Accordingly, the mobile application may use device or application specific interfaces to access the necessary athletic activity information. The APIs and/or other tools that may be used by third party developers may include security mechanism to prevent unauthorized access to athletic activity information.

Alternatively, or additionally, athletic performance data including, for example, miles run, calories burned, activity points earned and the like, may be transmitted between athletic performance monitoring devices such as device 201 and/or a remote athletic monitoring system. The transfer of data between devices may be performed through wired or wireless connections and networks including infrared connections, BLUETOOTH connections, wired and wireless local area networks, cellular networks, wide area networks such as the Internet and the like. The transmission of athletic information including activity points among performance monitoring devices, athletic activity data monitoring and tracking systems, non-athletic activity monitoring devices and other systems may be encrypted and/or transmitted using a secure or proprietary protocol to prevent unauthorized interception, access and/or tampering of such data. For example, Pretty Good Privacy (PGP) encryption may be used to encrypt athletic activity information. In another example, data may be transmitted using Secure Socket Layer (SSL) protocol to insure security. If the data is not encrypted according to an agreed-upon encryption algorithm or transmitted using a specified transmission protocol, the data may be rejected or communications in general may be denied.

Transmission and sharing of athletic activity information including activity points may allow a user to use multiple different athletic activity performance monitoring devices and/or other devices (e.g., personal computers, netbooks, mobile phones). For example, if a user uses different types of monitoring devices for different types of athletic activity, the user may wish to track an overall athletic activity level across all workouts and devices. Accordingly, the information collected and/or determined through each of the multiple devices may be shared and synchronized. However, in some configurations, activity point synchronization may require authorization through an activity point service to insure that activity points are not falsified, duplicated or modified in an unauthorized manner. For example, a user may be required to upload activity data to an activity point service from a first device and subsequently synchronize that data to each of one or more other devices the user owns through the activity point service. The activity point service may insure the legitimacy of synchronization between devices using such a configuration.

Data for other users might also be collected for a current user. For example, if a user wishes to track the athletic performance of a friend, the user may use his or her monitoring device to download or receive data for the friend. Such a process may require approval by the other user. This information, e.g., the type of information of the friend that the user views may also be used in determining user preferences, recommendations, suggestion; and/or customizing user experiences. For example, a user may frequently view details of a friend's soccer activities but not the friend's running workouts. In such an instance, a monitoring system may determine that the user is interested in soccer and less interested in running.

According to other aspects, a user's operation of a data collection and/or monitoring device such as device 201 (FIGS. 2A and 2B) or device 400 (FIG. 4) may be monitored and tracked. For example, the number of times a user presses a button or activates a function on the device may be tracked. Such information may be used by an athletic activity monitoring and tracking service to determine preferences by a user including activity preferences, data display preferences (e.g., a user may prefer to display split times versus overall times or heart rate rather than calories burned), storage preferences (e.g., users may consistently and/or frequently delete workout data that is more than a week old) and the like. Based on this information, a service or system may customize various aspects of the activity monitoring and tracking service. In one example, the service may limit displays of historical workout data to the past week if the service determines that users frequently delete workout data more than a week old from their monitoring device. In another example, the service may provide offers for products, services, events and the like that correspond to walking activities if the service detects that the user frequently uses a pedometer function of a device such as device 400 of FIG. 4. Other information that may be tracked based on usage of a device include preferred time of day or week for activities, preferred display configurations (e.g., split display versus whole displays), preferred information to be tracked (e.g., activity points versus calories burned) and the like.

The athletic activity data monitored and collected by a service may be used for a variety of purposes including the awarding and deducting of activity points. In one example, the data may be used to generate recommendations and/or suggestions for products, services, events, communities, articles and the like. In one example, a device may determine that the user has been exercising with the same pair of shoes for over 6 months. Accordingly, a system may recommend purchasing a new pair of shoes given the age of the current pair. The system may further recommend specific pairs of shoes or types of shoes based on the user's needs and/or preferences. For example, if sensors in a user's shoe indicate that the user has a pronation, the system may recommend shoes that are designed to correct for such a condition. In another example, if a new device that is an upgrade of a user's current device is available, the system may recommend or suggest purchasing the new device based on knowledge of the type of device the user currently uses. Alternatively, or additionally, recommendations for a new device may be made based on the features most frequently or recently used by the user.

Additional information may also be gleaned from the collected data including a health status of a user. For example, heart conditions, high or low blood pressure, stress levels, overall physical condition and the like may be determined based on heart rate measurements, pace information, calories burned, distance run and the like. Additionally or alternatively, one or more portions of the collected data may be used to determine an overall athleticism score.

In one or more arrangements, an activity monitoring and tracking service system may be used to configure monitoring devices such as monitoring device 201 (FIGS. 2A and 2B) and device 400 (FIG. 4). For example, a user may logon to a website or activate an application that allows a user to customize a look and feel of the user interface displayed on the monitoring device, types of information to be monitored, tracked and/or stored, user information including preferences and the like. In one or more arrangements, the information may be stored as a device profile and may be replicated to other devices. Accordingly, a user may configure multiple devices by propagating or copying the profile (e.g., if a user has different devices for different sports or if a user decides to upgrade from a first device to a second device). To customize the interface of the monitoring device, the application or website may replicate the device's interface to provide the user with a visual indication of the selections and changes being made. The application or website may further allow a user to define different interface options for different modes of the device (e.g., a clock mode versus a stopwatch mode). According to another aspect, the user may also customize the behavior of various buttons and functions of the device. Thus, a user may customize the actions performed by the device in response to pressing of a particular device button using the application or website.

### Activity Information Submission

As noted herein, activity points may be sponsored by an entity. For example, an athletic product company may sponsor a website or service that allows users to track and monitor their athletic activity. In addition, the athletic activity may be used to earn activity points as a type of currency that may be spent on a variety of events, services, privileges (e.g., VIP access) and products. Accordingly, users may need to submit athletic activity information to an athletic activity monitoring service before being awarded a corresponding number of activity points and being allowed to spend those points on rewards. While an estimate of a number of activity points earned or accumulated may be provided to a user on a mobile athletic performance monitoring device in real-time, the athletic performance information or other activity information may still need to be submitted to the monitoring service for final confirmation and validation.

In certain examples, activity points may be converted into various types of currencies, tokens, and the like, to be used to obtain actual or virtual products. For example, actual points may be converted to virtual tokens or cryptocurrency coins (e.g., which work through distributed ledger technology, such as a blockchain) to be spent on physical and/or virtual products. Rewards for a particular user profile may be shown in virtual points, but may additionally or alternatively be shown in terms of currencies, including money, coins, banknotes, as well as virtual currencies, including tokens, cryptocurrencies, central bank digital currencies, stablecoins, and the like. Validation of a virtual currency may be conducted in accordance with a plurality of different processes and/or systems, as would be appreciated by those skilled in the art. For example, a cryptocurrency may be validated as part of awarding cryptocurrency to a user account. Using a blockchain, a block may be appended to a hash that contains additional transaction information beyond a type and amount of virtual currency transferred by a merchant node, such as, for example, the value of the transferred virtual currency in generic virtual currency units along with a timestamp of the transaction. Each new block incorporating verified transactions may be added to the blockchain to determine when particular virtual currency units have been transferred or used and prevent double spending. As another example where virtual tokens are used, one or more token flags or electronic markers may be used. As would be appreciated by those skilled in the art, one or more flags may be set to one or more criteria. At least one criterion may be binary, such as whether the token is expired. In some examples, the provider specific tokens may be pushed to a blockchain and, after verification, may be added to the blockchain. For instance, a block may be added including the encrypted provider-specific token. The token added to the blockchain may then only be accessible to the respective provider who may decrypt the data and analyze the data to verify the token. Still in other examples, virtual points may be converted to currencies such as U.S. dollars, Euros, Japanese yen, British pounds, and the like.

Products available for purchase by a user may include any number of physical products such as athletic apparel, athletic footwear, wearable devices, athletic equipment, health/fitness tracking devices, user electronics and computing devices, and the like. Products available for purchase by a user may also include virtual items such as NFTs downloadable games, characters or avatars, virtual stickers, device applications, software upgrades, virtual game tokens, digital books, music, and movies, virtual courses or tutorials, printable files, coaching packages, event tickets, and the like.

### Process Flows

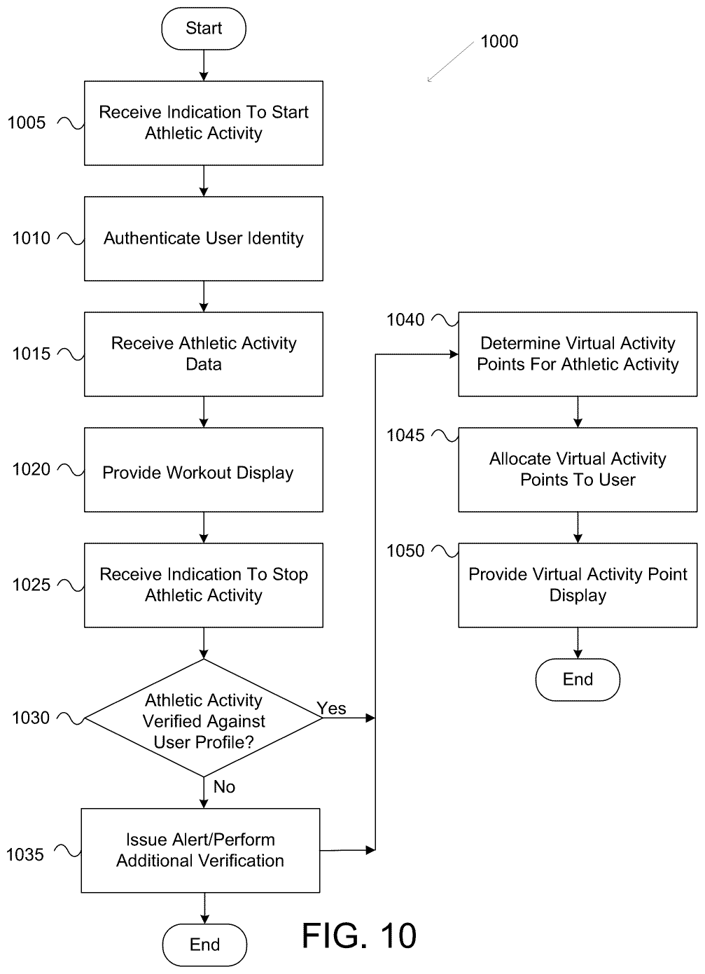

FIG. 10 illustrates a flowchart diagram of one or more processes for processing and analyzing user athletic activity data for a virtual point system in accordance with one or more aspects of the present disclosure. Processes described herein relate to processing and analyzing athletic activity data and providing a virtual reward system based on analyzed athletic activity data. In accordance with aspects of the present disclosure, apparatuses, systems, and methods described herein provide for efficient processing and analyzing of athletic activity data and motivational virtual reward systems based on analyzed athletic activity data. Some or all of the steps of process 1000 may be performed using one or more computing devices as described herein. In a variety of embodiments, some or all of the steps described below may be combined and/or divided into sub-steps as appropriate.

At step 1005, an indication to start an athletic activity may be received. Upon receiving the indication at step 1005, a user profile associated with the user in an athletic activity virtual point system may be identified. In some instances, the indication to start the athletic activity may be triggered by a user interacting with a device, such as device 112. In some examples, the indication to start the athletic activity may be triggered automatically at a time preset by the user or an athletic tracking program and/or in response to a user confirming that an athletic activity will be starting at a particular time. For example, step 1005 may occur upon the user interacting with a prompt to begin an athletic activity. In certain examples, upon receiving the prompt or indication, a number of virtual activity points in the associated user profile may be compared to an activity point cap to determine if the user has exceeded a cap on virtual activity points. Upon determining that the user has exceeded the cap, a notification may be issued to the user indicating that virtual activity points will not be allotted for that particular workout. In certain examples, upon receiving the prompt or indication, a schedule associated with that user profile may be analyzed to determine the user has missed a scheduled activity. Upon determining that the user has missed a scheduled activity, a prompt may be issued to the user for the user to perform one or more replacement activities that are selected based on being associated with a potential virtual point allotment in proportion to a potential virtual point allotment of the missed scheduled activity.

At step 1010, the user identity may be authenticated. In some instances, the user identity may be authenticated before the user begin the athletic activity. Additionally, or alternatively, the user identity may be authenticated at one or more points during the athletic activity and/or upon completion of the athletic activity. In some examples, the user identity may be authenticated using one or more image recognition processes. For instance, authenticating the user may include presenting a photo prompt to a display of device 112, receiving a photo responsive to user interaction with the photo prompt, and using an image recognition algorithm to verify that the photo matches image characteristics of the user associated with the user profile within a threshold amount. In other examples, the user identity may be authenticated by prompting the user present at the device 112 for a password or passcode, or to perform a 2-step verification using an additional verified device. In some instances, the user identity may be verified via one or more biometric devices worn by the user that are capable of identifying a personal characteristic of a user.

At step 1015, athletic activity data may be received as the user performs an athletic activity. In some examples, the process may only proceed to step 1015 upon successfully authenticating the user identity at step 1010. In other examples, the process may proceed to step 1015 without authenticating a user identity. The athletic activity data may be received at various intervals or continuously throughout the athletic activity and/or upon completion of the athletic activity. The athletic activity data may be transmitted from an activity monitoring device, such as device 112, device 126, and/or device 128, over a wired or wireless network in any number of ways, as described herein.

At step 1020, a workout display may be provided on the device 112 and/or computing device 110 as the user performs the athletic activity. In some examples, information provided as part of the workout display may include approximations to be verified and/or finalized at a later point in the process. For example, the workout display may include an estimate of a number of virtual points accumulated at a present point of the athletic activity, while the actual number of virtual points will be computed at a subsequent step in the process, as well be discussed in more detail below.

At step 1025, an indication to stop the athletic activity may be received. In some instances, the indication to stop the athletic activity may be triggered by a user interacting with a device, such as device 112. In some examples, the indication to stop the athletic activity may be triggered automatically at a time preset by the user or the athletic tracking program and/or in response to a user confirming that an athletic activity will be stopping after at a particular duration.

At step 1030, the athletic activity data may be verified against a user profile to verify that the athletic activity aligns with activity actually performed by the user associated with the user profile. In some instances, verifying the athletic activity may include determining that the athletic activity data falls within one or more thresholds associated with the user that are determined based on user profile information. In certain examples, verifying the athletic activity data may include training a machine learning model to determine the one or more thresholds for the user based on a plurality of user input parameters. In some examples, verifying the athletic activity data may include training a machine learning model to identify anomalous activity associated with the user profile based on at least the user profile information. In some instances, verifying the athletic activity data may include, upon determining that the activity information falls outside of the one or more thresholds associated with the user, determining a likelihood of anomalous data based a type of anomaly in the athletic activity data and/or an extent to which the athletic activity data falls outside of the one or more thresholds.

If the athletic activity data cannot be verified with the user profile, at step 1035, an alert may be issued to one or more computing devices, such as computing devices 110 and device 112, and/or a prompt to perform additional verification may be transmitted. In some examples, if additional verification is performed at step 1035, and the athletic activity data is verified by the additional verification, then the process may proceed to step 1040. In some instances, where the process proceeds to step 1035 upon determining that the athletic activity data falls outside of one or more thresholds associated with the user and upon determining that the athletic activity data is indicative of an anomaly relative to the user profile, at step 1035 an alert may be issued indicating that the athletic activity data is indicative of an anomaly and prompting the user for a follow up or additional verification. In some examples, where the process proceeds to step 1035 upon determining that the athletic activity data falls outside of one or more thresholds associated with the user but where the athletic activity data is not indicative of an anomaly relative to the user profile, the process may still proceed to step 1035, e.g., prompting the user for a follow up or additional verification, or the process may simply proceed to step 1040.

If the athletic activity data is verified for the user profile, at step 1040, an amount of virtual activity points associated with the athletic activity may be determined. In one or more instances, determining an amount of virtual activity points may include determining one or more parameters associated with the athletic activity selected from: a level of effort by the user while performing the first athletic activity, a type of activity of the first athletic activity, and a time for the user to complete the first athletic activity. Based on the one or more parameters associated with the athletic activity, a conversion rate may be selected from a plurality of rates for converting athletic activity data to virtual activity points. In some instances, determining an amount of virtual activity points may include determining a level of effort by the user while performing the athletic activity, and comparing the level of effort by the user while performing the athletic activity to a baseline effort of the user, e.g., based on one or more user profile information components, based on a current point in an athletic training program, and the like. In some examples, the baseline effort of the user may be determined by training a machine learning model based on data from the user as the user performs one or more previous athletic activities and/or based on user profile information. In some examples, the level of effort by the user may be determined based on one or more measurements taken during the activity, including but not limited to: speed, pace, resistance, duration, laps, weight, repetitions, elevation gain, improvement percentage in splits, heart rate, cadence, calories, and the like. Accordingly, the higher a user's level of effort is determined to be, the larger amount of virtual points may generally be awarded to that user.

At step 1045, the virtual activity points may be allocated to the user profile to which the athletic activity data was verified. Subsequently, at step 1050, a virtual activity point display may be provided, e.g., showing the virtual activity points accumulated from the athletic activity data and one or more aspects of the athletic activity. The virtual activity point display may include a detailed activity point breakdown including with one or more parameters associated with the athletic activity, one or more sections of the athletic activity, and allotted virtual points for each of the one or more sections. In one or more instances, the virtual activity points of the user profile may be converted to a currency, and a marketplace display may be presented with an indication of virtual points for items in the marketplace display. The currency may include any number of currencies, including virtual blockchain currencies. Additionally, the products (including virtual products such as NFTs) available for purchase may be displayed along with a required number of virtual points, actual currency, or blockchain currency for the purchase of each item. Upon receiving a user interaction with an item in the marketplace display, an item order for the user may be transmitted and an associated amount of virtual activity points from the user profile may be subsequently removed.

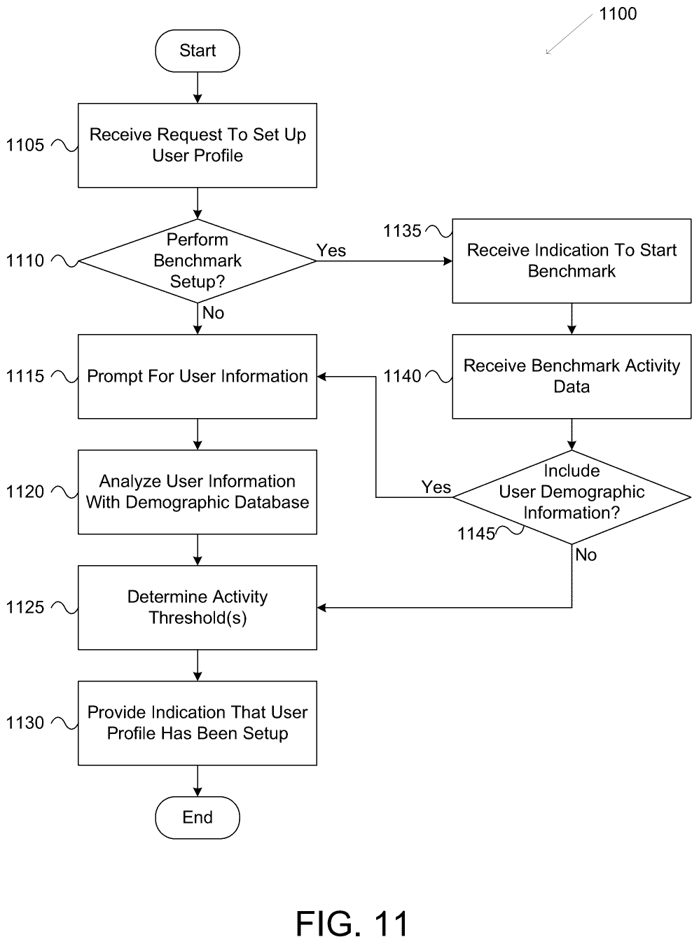

FIG. 11 illustrates a flowchart diagram of one or more processes for setting up a user profile as part of a virtual point system in accordance with one or more aspects of the present disclosure. Some or all of the steps of process 1100 may be performed using one or more computing devices as described herein. In a variety of embodiments, some or all of the steps described below may be combined and/or divided into sub-steps as appropriate.

At step 1105, a request to set up a user profile may be received. The request to set up the user profile may be associated with an athletic activity virtual point system, and/or may include one or more components of user profile information. Upon receiving the request at step 1105, the system may verify that there no existing user profile associated with the user information provided. In some instances, the request to set up the user profile may be triggered by a user interacting with a device, such as device 112. In some examples, the request to set up the user profile may be triggered automatically, e.g., by an athletic activity virtual point system being downloaded to the user device or by setting up a device verified with the athletic activity virtual point system.

At step 1110, the system may determine whether a benchmark setup is to be performed. In some instances, the benchmark setup may be performed in response to the user interacting with the user computing device to initiate the benchmark setup. In some examples, the benchmark setup may automatically be performed based on receiving one or more preset types of user information with the request to setup the user profile, or based on not receiving one or more types of information with the request to setup the user profile. Additionally, or alternatively, a benchmark setup may be performed after a user profile has been initially setup, e.g., where a user previously set up a user profile without a benchmark setup and now wishes to update the user profile and/or where a user has progressed through at least a portion of a training program and now wishes to update the benchmark activity dataset. In some examples, the user identity may be authenticated using one or more image recognition processes. In some instances, a benchmark setup may be performed or a type of benchmark setup may be selected based on one or more component of user information received with the request to set up the user profile, such as a type of activity to be performed, a subjective categorization of athletic fitness of the user, an indication that the user has previously completed another training program or fitness milestone, and the like.

If a benchmark setup is to be performed, the process may proceed to step 1135, where an indication to start the benchmark activity is received. In some instances, the indication to start the benchmark activity may be triggered by a user interacting with a device, such as device 112. In some examples, the indication to start the benchmark activity may be triggered automatically based on sensing physical movement at a user device, or at a time preset by the user or an athletic tracking program and/or in response to a user confirming that the benchmark activity will be starting at a particular time. For example, step 1135 may occur upon the user interacting with a prompt to begin the benchmark activity. In certain examples, a type of benchmark activity may be selected as part of step 1135, e.g., based on a selection by the user and/or one or more components of the user information such as a physical fitness level of the user, a recently completed fitness milestone, a type of training program to be performed, demographics of the user, and the like. Various type of benchmark activities may be available with varying time durations, indicated intensities, suggested equipment, and the like, which the user may select in advance, and/or which may be determined in response to one or more components of user information.

At step 1140, benchmark activity data may be received as the user performs the benchmark activity. The benchmark activity data may be received at various intervals or continuously throughout the benchmark activity and/or upon completion of the benchmark activity. The benchmark activity data may be transmitted from an activity monitoring device, such as device 112, device 126, and/or device 128, over a wired or wireless network in any number of ways, as described herein.

Upon completing the benchmark activity and receiving the processing the benchmark activity data, the process may then determine in user demographic information is to be additionally included. For example, if some user information was received as part of step 1105, step 1145 may include determining if additional user information is desired or if the user has indicated wanting to include additional user information to more completely setup a user profile. If user demographic information is to be included, the process may proceed to step 1115, otherwise the process proceeds to step 1125, as will be discussed in more detail below.

Upon determining that a benchmark setup will not be performed at step 1110 or upon completing the benchmark setup and determining that user demographic information will be included at step 1145, the process may proceed to step 1115 where a prompt for user information is transmitted to the device. In some instances, step 1115 may include analyzing what user information, if any, was received as part of step 1105 and determining additional components of user information that are missing. In some instances, the prompt at step 1115 may show information already received or determined as well as one or more prompts showing missing information and requesting the user to add the missing information. In some instances, the prompt at step 1115 may indicate one or more components of user information as mandatory to be included in order to setup a user profile and/or one or more components of user information as optional or preferred to be included in setting up the user profile.

At step 1120, the user information may be analyzed with information from a database of a plurality of users across various different demographics. In some instances, the user information analyzed at step 1120 may receiving a plurality of user input parameters, including one or more of: age, sex, height, weight, medical issue, pace, heart rate, athletic equipment identification, target athletic goal, and personal record.

Based on the analysis at step 1120, one or more activity thresholds for the user may be determined at step 1125. The one or more thresholds for the user may be determined based on the benchmark activity data received at step 1140. Additionally, or alternatively, the one or more thresholds for the user may be determined based on at least one of the plurality of user input parameters received at step 1115 and analyzed at step 1120. In some instances, determining the one or more thresholds may include training a machine learning model to determine the one or more thresholds for the user based on the plurality of user input parameters and accumulated data of a plurality of users across various user demographics. The one or more thresholds for the user may be updated based on activity information as the user performs one or more athletic activities.

Subsequently, at step 1130, an indication that the user profile has been setup may be provided on the user device. The indication may include a number of parameters received as part of the user profile setup process, one or more thresholds determined during the setup process, a suggested athletic workout schedule for the user, and the like. In some instances, the indication may include one or more elements that the user may interact with to provide additional information and/or further refine one or more components of the user profile.

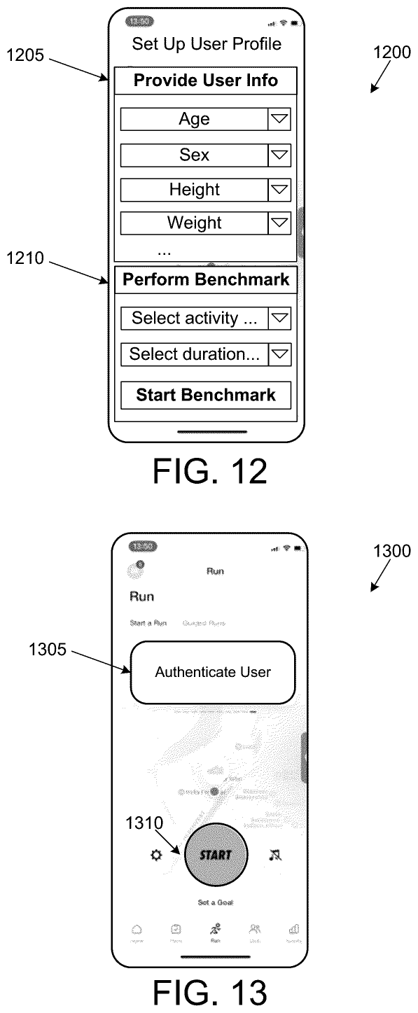

FIGS. 12-17 illustrate various user interfaces of a virtual point system in accordance with one or more aspects of the present disclosure.

FIG. 12 illustrates an example of a user setting up a user profile for an athletic activity performance monitoring service. In FIG. 12, for example, an interface 1200 may be provided based on a user interacting with an athletic activity performance monitoring interface to begin a user profile setup or based on an athletic activity performance application being downloaded to a user device. As shown in interface 1200, a user information portion 1205 may be displayed and may be interacted with to provide various demographic information relating to the user. The information provided in user information portion 1205 may include a plurality of user input parameters, including one or more of: age, sex, height, weight, medical issue, pace, heart rate, athletic equipment identification, target athletic goal, and personal record. Upon interacting with the user information portion 1205, process similar to that described with respect to steps 1115 and 1120 in FIG. 11 may be initiated. A benchmark portion 1210 may be displayed and may be interacted with to provide initiate a benchmark activity as part of a user profile setup. Upon interacting with the benchmark portion 1210, process similar to that described with respect to steps 1135 and 1140 in FIG. 11 may be initiated.

FIG. 13 illustrates an example of a user initiating the start of an activity showing portions of an athletic activity performance monitoring interface relating to starting an activity and/or authenticating the identity of a user. In FIG. 13, for example, an interface may be provided based on a user interacting with an athletic activity performance monitoring interface or based on a preset condition occurring to trigger an interface for a user to start an activity. As shown in interface 130o, a user authentication prompt 1305 may be displayed and may be interacted with to authenticate the user. Upon interacting with the user authentication prompt 1305, process similar to that described with respect to step 1010 in FIG. 10 may be initiated. In some examples, the user identity may be authenticated using one or more image recognition processes. For instance, authenticating the user may include presenting a photo prompt, receiving a photo responsive to user interaction with the photo prompt, and using an image recognition algorithm to verify that the photo matches image characteristics of the user associated with the user profile within a threshold amount. In other examples, the user identity may be authenticated by prompting the user for a password or passcode, or to perform a 2-step verification using an additional verified device. In some instances, the user identity may be verified via one or more biometric devices worn by the user that are capable of identifying a personal characteristic of a user. In some instances, the user identity may be authenticated before the user begin the athletic activity. Additionally, or alternatively, the user identity may be authenticated at one or more points during the athletic activity and/or upon completion of the athletic activity.

Additionally, a workout start button 1310 may be provided and the user may interact with the workout start button 1310 to initiate a workout. In some examples, the workout start button 1310 may only be selectable after the user has been authenticated by interacting with user authentication prompt 1305. In some examples, the workout start button 1310 may selectable even if the user has not been authenticated. In some instances, if the user selects the workout start button 1310 without first being authenticated, the a user authentication prompt may occur during the workout or upon completion of the workout. In some examples, authenticating the user before starting the workout may result in a larger conversion rate of activity points in order to motivate the user to perform the authentication process before starting the workout. By first authenticating the user, the system better ensures that a user will not “cheat” the system by having other perform activities to rack up points for that user.

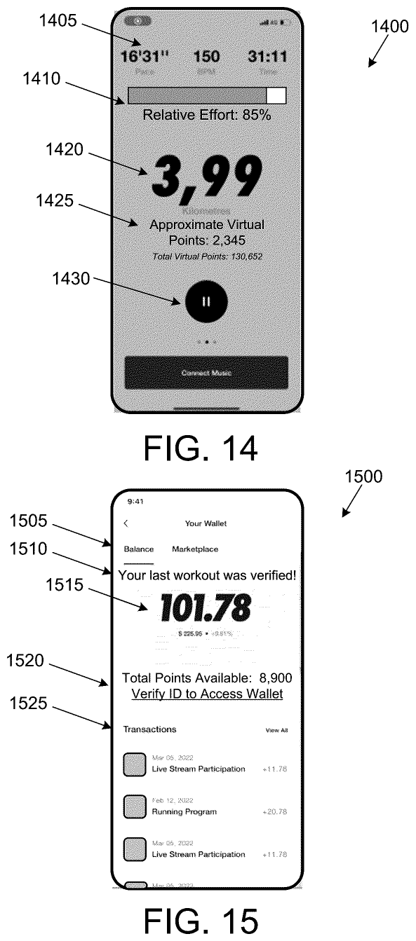

Activity information earned activity points may be displayed for user review and management in a variety of manners. FIG. 14 illustrates an interface 1400 (e.g., of a display of athletic information monitoring device 201 of FIGS. 2A and 2B and/or device 112 of FIG. 1) configured to convey athletic performance information before, during and/or after a workout. For example, in interface 1400, athletic activity measures including, for example, a number of kilometer or miles covered, pace, calories burned, heart rate, amount of time, amount of force used (e.g., in weightlifting or step exercises), steps taken and/or combinations thereof may be displayed in a first portion 1405. A relative effort by the user may be displayed simultaneously or sequentially with the relative effort portion 1410. A summary parameter may be displayed in a larger font in a second portion 1420. For example, total distance covered may be displayed in a second portion 1420 of interface 1400. A third portion 1425 of interface 1400 may display information relating to a user's virtual point allotment. For example, an approximate amount of virtual points amassed during a present workout may be displayed above a total number of virtual points currently associated with the user profile. The information displayed in the first portion 1405 and second portion 1420 may depend on the type of workout in which the user is currently engaged and/or the information that the user has selected for display as an athletic activity measure in the second portion 1420. For example, if the user elects to display an amount of time run or exercised, portion 1420 may display a current time duration of the present activity and/or the greatest amount of time the user has ever exercised (or within a predefined amount of time such as the last month, among all recorded data, last year, last week, last 10 workouts, etc.) or a current goal that the user is attempting to accomplish or reach. An activity pause button 1430 may be provided in the interface 1400. Interaction with the activity pause button 1430 may cause the activity to temporarily pause (and to temporarily stop receiving activity data), and further interaction may end the current activity or restart the current activity. The type of workout may also be identified in interface 1400, e.g., with a descriptor and/or an icon representing the activity.

The user may configure interface 1400 in a variety of ways including selecting the types of information displayed therein. In one example, the user may elect to display a corresponding or estimated number of activity points in third portion 1425. Activity points displayed in third portion 1425 in interface 1400 may only be an estimate since the number of activity points awarded may require final verification and analysis. For example, analysis to verify that the activity is align with activity associated with the user profile and a determination of a relative effort expended by the user during the activity may influence a final determination of the number of points to be awarded based on the user's workout information. Activity points may correspond to a total number of activity points of a particular type or grade, available to a user (e.g., unspent activity points accumulated from a current workout in addition to previous workouts), or accumulated during the current workout session. One or more aspects of interface 1400 may be modified and configured according to user preferences. For example, a user may choose to turn off the activity point tracker in third portion 1425. In another example, a user might not wish to display the relative effort portion 1410. In other examples, a user may rearrange the placement, size and appearance of various information portions to suit the user's viewing preference.

The appearance of information displayed in a user interface such as interface 1400 of FIG. 14 may change depending on one or more variables of a workout. For example, if a user enters a heart rate range that is at or above 90% of the user's resting heart rate, the displayed heart rate may change color, font, size and/or combinations thereof. In another example, a workout metric may change color upon reaching or approaching various targets (e.g., miles, calories, steps), exceeding a personal best, exceeding or falling below a target pace, etc. Other portions of an interface (e.g., beyond the workout metric) may also change in appearance. In one example, the entire interface may flash or rapidly alternate between two colors upon detecting a triggering event. For example, a user's heart rate may be displayed in an alternate color and in an increased font size upon detecting the user's heart rate exceeding a predefined limit. In contrast, when a user's heart rate is below the predefined limit, the user's heart rate may be displayed in green or black and in decreased font size. Alternatively, or additionally, the heart rate may blink or flash to further alert the user to the detected condition. If the user's heart rate drops below a second threshold, the user may be similarly alerted using a change in color, font size, font or the like. This may help alert the user to when he or she is outside of a preferred exercise heart rate range. Other appearance characteristics may also be modified including font style (e.g., bold, italic, underlined), capitalization (all caps, small caps), background color, text and/or graphic position and the like, and for any number of parameters displayed in interface 1400.

As another example in which a portion of interface 1400 may change in appearance upon the occurrence of a triggering event a target activity point may be set. A user, for instance, may set the target activity point. This target activity point may correspond, in one or more examples, to a number of activity points required to obtain a particular reward or to achieve some other goal. In one or more examples, the goal or reward (not shown) that the user is trying to achieve may be displayed or represented in interface 1400. Alternatively, the target activity point level may be a maximum amount of activity points that may be gained or earned over a predefined time period (e.g., at any one time, per day, per week, per month, per workout). In some examples, portions of the interface 1400 may change to a first color upon a user reaching a target or threshold level of activity points. Activity point portion shown in the third portion 1425 may further change to a second color upon the number of activity points reaching 75% of the target level and yet another color upon reaching 100% of the target level. Various thresholds for modifying the appearance of third portion 1425 may be defined depending on user preferences, system default settings, activity point provider/sponsor specifications and/or combinations thereof.

While FIG. 14 shows the activity points in the third portion 1425 as a listed number, the activity points may be displayed in a number of additional formats. For example, though not shown in FIG. 14, activity points may be displayed using a graphical icon resembling a battery gage. As a user earns activity points, a display of the battery gage may be updated to show it filling up. The color of the battery gage contents may change depending on the current fill level of the battery gage. For example, the battery gage contents may be displayed in red when the fill level is at or below 25%, yellow when the fill level is above 25% and below 75% and green when equal to or above 75%. When the battery gage is entirely filled, the gage contents may change to yet another color such as blue or black. Alternatively, sections of the battery gage may be displayed in different colors. That is, a portion between 0% and 25% may be displayed in red while a portion between 25% and 50% may be displayed in yellow. Other types of representation may be used to display the level of activity points achieved or earned. For example, a user's current level of activity points may be represented using a fuel gage (e.g., a fuel needle pointing between empty and full), a thermometer-type gage, a pie chart and the like.

Upon completing a workout, a user may be presented with a post-workout activity point display, that may include activity points from the most recent activity or activities as well as a summary of total activity points associated with a user profile. Accordingly, when a user wishes to review the amount of activity points earned, an interface may be generated where multiple activity point summaries may be displayed. For example, FIG. 15 illustrates a user interface 1500 in which activity point summaries are provided. For example, the interface 1500 may include a top portion 1505 that allows a user to toggle between viewing a balance of activity points and a marketplace of products or services available for the user to purchase using the activity points, as will be described in more detail below. The interface 1500 may also include a verification notification 1510 indicating that the most recent workout was verified as having been performed by the user associated with the user account. Additionally, a last workout point display 1515 may be provided in interface 1500 showing the final number of verified activity points attributed to the last workout. The interface 1500 may also include a summary portion 1520 including a summary of the total number of activity points available. A selectable link may also be provided in the summary portion 1520 to verify a user's identification in order to access the full wallet of activity points. The interface 1500 may further include a point history portion 1525 with a summary of older workout and related activity points earned int hose workouts.

In some arrangements, activity points of different grades or value may be tracked and visually represented in a single activity point interface. However, to allow a user to differentiate between the grades (and/or the modes through which the activity points were earned), the contents of an activity point gage may be displayed in different colors in accordance with a proportion of activity point grades. The accumulated activity points of any particular grade may be merged into a single continuous portion of the gage or may be allocated in the gage according to a time at which the points were earned. Hovering, clicking or otherwise interacting with a an activity point summary, e.g., last workout point display 1515 or an older workout entry in point history portion 1525 of FIG. 15 may cause information and details about the activity points and/or athletic activity performed to be displayed. For example, a detailed information bubble may provide information regarding the selected workout. Additionally, a total exercise or workout time may also be displayed.

Activity points and activity related information may also be displayed in one or more widgets or applications. For example, a user may display widgets on a social network application that can receive athletic activity information from an athletic activity performance monitoring service. The social network application may provide live feeds of such information or may retrieve activity data on a predefined schedule. Alternatively, a user may manually request updating of the activity data. In additional or alternative arrangements, applications or widgets may be downloaded to a user device (e.g., other than an athletic activity performance monitoring device), so that the user may view his or her athletic activity or other activity information without accessing a remote network site or using an athletic performance monitoring device. As discussed herein, the widgets or applications may be developed by third party developers and require access to proprietary protocols or interfaces.

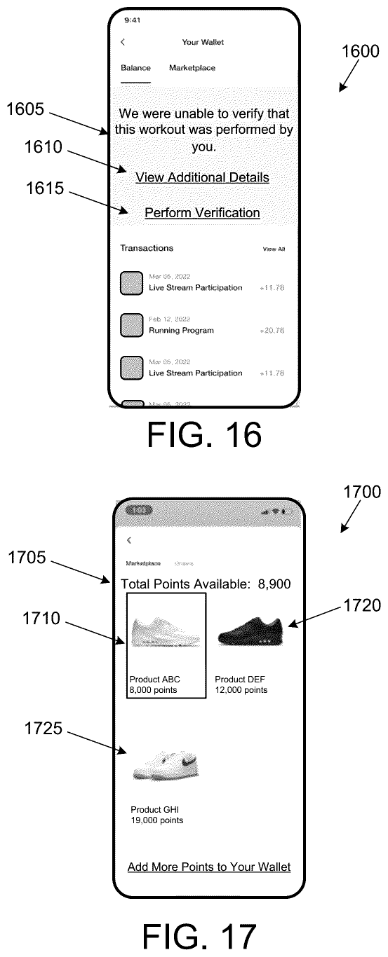

FIG. 16 illustrates another example of an interface displaying activity points. In interface 1600, the most recent workout was not verified as having been performed by the user associated with the user account. As such, the interface 1600 may include a verification message 1605 that indicated that the previous workout was not able to be verified. The interface 1600 may also include an additional details link 1610 that selectably allows the user to view additional details associated with the last workout and/or data relating to the failure to verify the last workout with the user profile. For example, interaction with the additional details link 1610 may cause a graphical display to be shown that includes an indicator of data from the last workout relative to one or more threshold associated with the user profile to graphically indicate that the last workout is anomalous for the user profile. As another example, activity points may be interaction with the additional details link 1610 may cause a text-based display showing a number of parameters associated with the most recent workout and an indicator of which of the parameters are anomalous for the user profile. The interface 1600 may also include an additional details link 1610 that selectably allows the user to perform an additional verification related to the most recent workout. The additional verification may include one or more steps similar to the initial verification and/or may include varying additional steps, such as prompting a user to provide certain information or to authenticate the user's identity or to repeat an activity so as to achieve a similar metric to the anomalous most recent workout.

FIG. 17 illustrates an example interface in which a user's accumulated activity points, activity information and activity information submission options may be displayed and accessed. Interface 1700 may include an available point portion 1705 that displays a total number of activity points accumulated by and available to the user. As illustrated, a single total of activity points is shown. In other examples, the available point portion 1705 may include a plurality of different grades of activity points. Similar to differences in currency, the activity points represented in a first grade of point may be more valuable than the activity points represented in a second grade of points.

Interface 1700 may display reward information and allow a user to browse various rewards that are achievable through the use of activity points. For example, interface 1700 may include a scrollable listing of rewards 1710, 1720, 1725 that may be purchased. Rewards that are within a user's available point range (reward 1710) may be displayed in a first manner (e.g., in color and/or with visual details) while rewards that are not obtainable (rewards 1720 and 1725) based on a user's currently available number of points may be displayed in a second manner (e.g., in an outline form, in black & white, grayed out, faded, etc.). The rewards 1710, 1720 1725 displayed in interface 1700 may be selected in a variety of ways including based on a user's interests. A user's interests may be determined based on a history of user activity including types of workouts, products or services purchased, events attended, services subscribed to/purchased and the like. Upon selecting one of the rewards, the user may be provided with a details option to view additional information about the reward as well as a purchase option (if the user has sufficient points). While the rewards shown in the interface 1700 include physical items for purchase, virtual items such as NFTs downloadable games, characters or avatars, virtual stickers, device applications, software upgrades, virtual game tokens, digital books, music, and movies, virtual courses or tutorials, printable files, coaching packages, event tickets, and the like, may additionally be displayed as being available for purchase in the interface 1700. Additionally, while the rewards shown in the interface 1700 as well as the reward 1710 as shown in virtual points, rewards may be shown in terms of currencies, including money, coins, banknotes, as well as virtual currencies, including tokens, cryptocurrencies, central bank digital currencies, stablecoins, and the like.

According to one or more aspects, though not shown in FIG. 17, interface 1700 may include a portion that displays a user goal and a number of points needed to achieve goal. A user may be able to modify goal by selecting an option and manually specifying a goal or choosing a new goal from a menu. An activity record may also be included in interface 1700, which may include a running entry of workout information and a corresponding number of activity points allotted for each activity and/or other activity information such as most recent shopping event, a number of advertisements reviewed, an article read, and the like. Additionally, each activity entry may include an option that allows the user to view detailed information relating to that activity. For example, the detailed information may include a breakdown of the activity point calculation, heart rate information, a relative level of effort by the user, a breakdown of purchases made at a retailer and the like.

Interface 1700 may further allow a user to manage activity points by purchasing or selling activity points. For example, an option bar may allow a user to purchase additional activity points if the user would like to purchase a reward immediately but does not have a sufficient number of points. Limits may be on a per day basis, per week, per month, per year and the like. Additionally, or alternatively, the cost of points may be different depending on one or more components of the user profile. For example, the costs of activity points may become lower if a user's athletic activity level improved. Conversely, if a user's athletic activity declines or is low, the cost of activity points may be higher or may be increased. Other attributes and/or characteristics of a user may also affect the cost of activity points such as a number of athletic events in which the user has participated, a number of friends recruited to use the service and/or an athletic activity level trend.

Activity points may be consumed or recognized in a variety of manners. As noted above, users may spend activity points on a variety of items including services such as VIP treatment at stores, events, flights, hotels and other venues; product and/or service discounts (e.g., coupons or free products); video game related items including game credits (e.g., to buy additional weapons or levels), in-game powers or skills, or players (e.g., unlocking a famous athlete); and the like. The activity point cost of these items may be defined by the athletic performance monitoring service and may fluctuate depending on popularity. For example, activity points may be used to purchase flights or concert tickets. In one or more arrangements, a user may select one or more particular interests. Those interests may then be used to modify activity point costs of items relating to those interests. For example, if a user enjoys spa treatments, the user may be offered discounted services from spa treatments. A user may be restricted to a predefined number of interests that are reflected in the price of rewards. Alternatively, or additionally, various services, products or other rewards may be discounted if a user allows advertisements to be displayed in his or her athletic performance monitoring site and/or if the user views a certain number of advertisements over a specified time period. For example, a user may allow the placement of advertisements in various locations of the user's site. Accordingly, the sponsors of those advertisements may offer discounts to that user for rewards that they provide through the athletic performance monitoring service. Alternatively, the athletic performance monitoring service may provide discounts based on advertisement revenue received from sponsors when users visit a specified number of advertisements.

Activity points may be consumed or spent in additional or alternative ways. For example, a user may wish to donate points to one or more charities or other organizations, events, services and the like. The donations may be tax-deductible based on an equivalent monetary value. Additionally, or alternatively, a user may transfer or sell activity points to other users. For example, a user may gift a certain number of points to another user. A user may be required to pay a premium or other cost for transferring points to another user whereas a lower or no premium or cost may be associated with making donations to charitable organizations. If a user decides to sell his or her activity points, the service may further charge the user a percentage of the sales price, a listing fee or other transaction fee, sales tax and/or other added costs for the transaction. Limits may also be set by the service for the number of points that may be transferred between users, sold and/or donated. The limits may be defined in a dollar amount or by number of activity points. Limits may also be different depending on the type of transfer. For example, transfers between users may be limited to a first threshold (e.g., 500 points per month) while donations to charitable organizations may be unlimited. Points for sale, on the other hand, may be capped at 1000 points per week. Limits may also vary depending on the user. For example, users may subscribe to a premium service that allows higher or unlimited caps to points sold or transferred for free to other users. In another example, users with higher activity levels may be allowed higher limits for such transactions.

Activity points may also be awarded and deducted based on challenges, objectives and goals. For example, if a user completes an objective or goal, a number of activity points may be awarded. In another example, different numbers of activity points may be awarded for a challenge depending on a user's finish in the challenge. As noted herein, points might also be deducted if a user fails to complete a challenge, objective or goal. Challenges, objectives and goals may be generated by the user or issued by others including other athletes or coaches. In one or more arrangements, the user may explore and participate in various challenges by browsing through a challenge marketplace or community. Invitations may also be sent through social networking sites and/or using information feed services R. Users may be able to check on the status of a challenge by viewing a live leaderboard that indicates a current progress of each of the challenge's participants. In some arrangements, the users may be able to view the progress or leaderboard through their monitoring devices or another device such as a personal computer. Alternatively, or additionally, updates to challenge progress or status may be pushed to a user's device automatically.

Challenges, objectives and goals may also be suggested or recommended to the user by an athletic activity monitoring system. For example, the system may identify or generate challenges configured push a user to achieve a 10% gain in maximum distance run in one workout. Accordingly, the challenge may be defined using activity data collected from the user (in particular, a maximum distance run in a single workout). Challenges might also be selected or generated based on a user's known interests, characteristics and preferences. In one example, a user's athleticism rating may be used to select or generate appropriate challenges for a user.

Providing an activity environment having one or more of the features described herein provides a user with an immersive experience that will encourage and motivate the user to engage in athletic activities and improve his or her fitness. By providing the user with current that may be used to purchase real world items that may be unrelated to fitness or athletic activity, the user is further motivated to engage in those activities that reward additional currency. Users may further communicate through social communities and challenge one another to reach various levels of fitness. In addition to traditional athletic activity measures, the use of an activity currency allows users to view their fitness level and activity in a more practical manner.

Additionally, as products or services may be rewarded to users based on completed a requisite number of activities, users may be tempted to cheat the athletic monitoring and virtual activity point system by finding ways to amass points without actually performing the activities, e.g., by having friends perform the activity, or by logging a car trip as a run or bike ride. Various aspects described herein provide ways to mitigate such cheating and better enhance the experience and motivational aspects of the athletic monitoring and virtual activity point system, e.g., by verifying activity data, authenticating a user identity, and/or determining a relative effort level of the user performing an activity. Further by using various aspects of user data, e.g., benchmark data, user demographic and profile information, previous workout history data, the system may be updated over the course of an athletic training program and may better tailor the experience to the actual fitness level of the user.

One or more aspects discussed herein may be embodied in computer-usable or readable data and/or computer-executable instructions, such as in one or more program modules, executed by one or more computers or other devices as described herein. Generally, program modules include routines, programs, objects, components, data structures, and the like that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a hard disk, optical disk, removable storage media, solid-state memory, RAM, and the like. As will be appreciated by one of skill in the art, the functionality of the program modules may be combined or distributed as desired in various embodiments. In addition, the functionality may be embodied, in whole or in part, in firmware or hardware equivalents such as integrated circuits, field programmable gate arrays (FPGA), and the like. Particular data structures may be used to more effectively implement one or more aspects discussed herein, and such data structures are contemplated within the scope of computer executable instructions and computer-usable data described herein. Various aspects discussed herein may be embodied as a method, a computing device, a system, and/or a computer program product.

Although aspects of the present disclosure have been described in certain specific aspects, many additional modifications and variations would be apparent to those skilled in the art. In particular, any of the various processes described above may be performed in alternative sequences and/or in parallel (on different computing devices) in order to achieve similar results in a manner that is more appropriate to the requirements of a specific application. It is therefore to be understood that systems, methods, and apparatuses described herein may be practiced otherwise than specifically described without departing from the scope and spirit of the present disclosure. Thus, aspects of the present disclosure as described herein should be considered in all respects as illustrative and not restrictive.

What is claimed is:

1. A computer-assisted method comprising:
receiving, by a processor, a user request to set up a user profile for an athletic activity virtual point system, wherein the user request includes user profile information;
receiving, from an activity monitoring device associated with a user, activity information as the user performs a first athletic activity;
verifying the first athletic activity, wherein verifying the first athletic activity includes determining that the activity information falls within one or more thresholds associated with the user that are determined based on the user profile information;
upon verifying the first athletic activity, determining a first amount of virtual activity points to allocate to the user profile based on the first athletic activity;
allocating the first amount of virtual activity points to the user profile; and
providing a virtual activity point display showing the first amount of virtual activity points accumulated from the first athletic activity and one or more aspects of the first athletic activity.

2. The computer-assisted method of claim 1, wherein receiving user profile information includes receiving benchmark activity data, from the activity monitoring device, as the user performs a benchmark activity, and wherein the one or more thresholds for the user are determined based on the benchmark activity data.

3. The computer-assisted method of claim 1, wherein receiving user profile information includes receiving a plurality of user input parameters, wherein the plurality of user input parameters includes one or more of: age, sex, height, weight, medical issue, pace, heart rate, athletic equipment identification, target athletic goal, and personal record, and wherein the one or more thresholds for the user are determined based on at least one of the plurality of user input parameters.

4. The computer-assisted method of claim 3, further comprising training a machine learning model to determine the one or more thresholds for the user based on the plurality of user input parameters and accumulated data of a plurality of users across various user demographics, and wherein the one or more thresholds for the user are updated based on activity information as the user performs one or more athletic activities.

5. The computer-assisted method of claim 1, wherein verifying the first athletic activity includes training a machine learning model to identify anomalous activity associated with the user profile based on at least the user profile information.

6. The computer-assisted method of claim 1, furthering comprising authenticating the user associating with the user profile before the user begins the first athletic activity.

7. The computer-assisted method of claim 6, wherein authenticating the user includes:
presenting a photo prompt to a display of a user device;
receiving a photo responsive to user interaction with the photo prompt; and
verifying, using an image recognition algorithm, that the photo matches image characteristics of the user associated with the user profile within a threshold amount.

8. The computer-assisted method of claim 1, wherein verifying the first athletic activity includes, upon determining that the activity information falls outside of the one or more thresholds associated with the user, determining a likelihood of anomalous data based on at least one of: a type of anomaly in the activity information or an extent to which the activity information falls outside of the one or more thresholds.

9. The computer-assisted method of claim 1, wherein determining a first amount of virtual activity points includes:
determining one or more parameters associated with the first athletic activity selected from: a level of effort by the user while performing the first athletic activity, a type of activity of the first athletic activity, and a time for the user to complete the first athletic activity; and
selecting, based on the one or more parameters associated with the first athletic activity, a conversion rate from a plurality of rates for converting activity data to virtual activity points.

10. The computer-assisted method of claim 1, wherein determining a first amount of virtual activity points includes:
determining a level of effort by the user while performing the first athletic activity; and
comparing the level of effort by the user while performing the first athletic activity to a baseline effort of the user, wherein the baseline effort of the user is determined by training a machine learning model based on data from the user as the user performs one or more previous athletic activities and/or based on user profile information.

11. The computer-assisted method of claim 1, further comprising:
providing a workout display to a user device during the first athletic activity, wherein the workout display includes an estimate of activity points accumulated during the first athletic activity.

12. The computer-assisted method of claim 1, wherein the virtual activity point display includes a detailed activity point breakdown including with one or more parameters associated with the first athletic activity, one or more sections of the first athletic activity, and allotted points for each of the one or more sections.

13. The computer-assisted method of claim 1, further comprising:
upon the user interacting with a prompt to begin a second athletic activity, determining if the user has exceeded a cap on virtual activity points, and upon determining that the user has exceeded the cap, issuing a notification to the user indicating that virtual activity points will not be allotted for the second athletic activity.

14. The computer-assisted method of claim 1, further comprising:
converting virtual activity points of the user profile to a currency; and
presenting a marketplace display to a user device with an indication of virtual points for items in the marketplace display; and
upon receiving a user interaction with an item in the marketplace display, transmitting an item order for the user and removing an associated amount of virtual activity points from the user profile.

15. The computer-assisted method of claim 1, further comprising, upon determining that the activity information falls outside of one or more thresholds associated with the user, determining if the activity information is indicative of an anomaly relative to the user profile, and issuing an alert to a user device indicating that the activity information is indicative of an anomaly and prompting the user for a follow up verification.

16. The computer-assisted method of claim 1, further comprising:
determining if the user has missed a scheduled activity; and
issuing a prompt to a user device for the user to perform one or more replacement activities, wherein the one or more replacement activities are selected based on being associated with a potential virtual point allotment in proportion to a potential virtual point allotment of the missed scheduled activity.

17. A non-transitory computer-readable medium storage medium storing computer readable instructions that, when executed, cause a processor to perform a method comprising:
receiving, from an activity monitoring device associated with a user, an indication that the user is to perform a first athletic activity;
determining a user profile associated with the user for an athletic activity virtual point system;
receiving, from the activity monitoring device, activity information as the user performs a first athletic activity;
verifying the first athletic activity, wherein verifying the first athletic activity includes determining that the activity information falls within one or more thresholds associated with the user that are determined based on the user profile;
upon verifying the first athletic activity, determining a first amount of virtual activity points to allocate to the user profile based on the first athletic activity;
allocating the first amount of virtual activity points to the user profile; and
providing, to a display of the activity monitoring device, a virtual activity point display showing the first amount of virtual activity points accumulated from the first athletic activity and one or more aspects of the first athletic activity.

18. The non-transitory computer readable storage medium of claim 17, wherein verifying the first athletic activity includes training a machine learning model to determine the one or more thresholds for the user based on a plurality of user input parameters.

19. The non-transitory computer readable storage medium of claim 17, wherein verifying the first athletic activity includes, upon determining that the activity information falls outside of the one or more thresholds associated with the user, determining a likelihood of anomalous data based on at least one of: a type of anomaly in the activity information or an extent to which the activity information falls outside of the one or more thresholds.

20. A system comprising:
an activity monitoring device configured to capture activity data relating to an activity performed by a user; and
an electronic device in communication with the athletic activity monitoring, wherein the electronic device includes a processor and a non-transitory computer-readable storage medium storing computer-executable instructions that, when executed, cause a processor to perform a method comprising:
receiving, by the processor, user profile information as part of setting up a user profile for an athletic activity virtual point system;
receiving, from the activity monitoring device associated with the user, activity information as the user performs a first athletic activity;
verifying the first athletic activity, wherein verifying the first athletic activity includes determining that the activity information falls within one or more thresholds associated with the user that are determined based on the user profile information;
upon verifying the first athletic activity, determining a first amount of virtual activity points to allocate to the user profile based on the first athletic activity; and
providing, to a display of the electronic device, a virtual activity point display showing the first virtual activity points accumulated from the first athletic activity and one or more aspects of the first athletic activity.
